(19) United States
(12) Patent Application Publication
(10) Pub. No.: US 2010/0015125 A1
(43) Pub. Date: Jan. 21, 2010

(54) METHODS AND COMPOSITIONS FOR ENHANCING IMMUNE MEMORY BY BLOCKING INTRAHEPATIC ACTIVATED T CELL DELETION

(75) Inventors: Ian Nicholas Crispe, Penfield, NY (US); David Topham, Pittsford, NY (US); Beena John, Rochester, NY (US); Ingo Klein, Rochester, NY (US)

Correspondence Address: NIXON PEABODY LLP - PATENT GROUP, 1100 CLINTON SQUARE, ROCHESTER, NY, 14604, US

(73) Assignee: UNIVERSITY OF ROCHESTER, Rochester, NY (US)

(21) Appl. No.: 11/917,866

(22) PCT Filed: Jun. 19, 2006
(86) PCT No.: PCT/US06/23682

§ 371 Date: Oct. 2, 2009

Related U.S. Application Data
(60) Provisional application No. 60/691,575, filed on Jun. 17, 2005.

(51) Int. Cl. A61K 39/395; A61K 31/7088; A61K 38/00; A61K 39/00

(52) U.S. Cl. 424/130.1; 514/44 A; 514/12; 424/184.1

(57) ABSTRACT

The present invention discloses a method of inhibiting CD8+ T cell deletion by the liver via the use of Toll-like receptor-4 inhibitors. Also disclosed are compositions of Toll-like receptor-4 inhibitors and either immunogenic agents or activated CD8+ T cells, which can be used to enhance secondary immune responses in normal and immunocompromised subjects. The administration of Toll-like receptor-4 inhibitors, alone or in combination with one or both of immunogenic agents or activated CD8+ T cells, to subjects to enhance secondary immune responses is also disclosed.

This application claims the benefit of U.S. Provisional Patent application Ser. No. 60/691,575, filed Jun. 17, 2005, which is hereby incorporated by reference in its entirety.

This application was made, at least in part, with funding received from the National Institutes of Health under RO1 grants AI037554 and AI063353. The U.S. government may retain certain rights in this invention.

### FIELD OF THE INVENTION

The present invention is directed to methods and compositions for enhancing immune cell memory by blocking intrahepatic activated T cell deletion via Toll-like receptor-4 regulation.

### BACKGROUND OF THE INVENTION

The ability to respond to a pathogen more vigorously upon second exposure is a cardinal feature of the adaptive immune system, and the principle underlying vaccination. Upon initial exposure to antigen, CD8+ T cells go through massive clonal expansion followed by dissemination of these cells to various tissues (Klonowski et al., “The CD8 Memory T Cell Subsystem: Integration of Homeostatic Signaling During Migration,” Semin Immunol 17:219-29 (2005); Seder et al., “Similarities and Differences in CD4+ and CD8+ Effector and Memory T Cell Generation,” Nat Immunol 4:835-42 (2003)). After clearance of the antigen, there is a contraction phase, which involves large scale CD8+ T cell apoptosis (Murali-Krishna et al., “Counting Antigen-Specific CD8 T Cells: A Reevaluation of Bystander Activation During Viral Infection,” Immunity 8:177-87 (1998)). According to the linear model of differentiation, a small population of effector CD8+ T cells survives this elimination process and is the source of a stable memory population (Seder et al., “Similarities and Differences in CD4+ and CD8+ Effector and Memory T Cell Generation,” Nat Immunol 4:835-42 (2003)). The role of different factors during the priming and contraction phase of CD8+ T cells in regulating the size of the memory T cell pool has been extensively studied (Kaech et al., “Selective Expression of the Interleukin 7 Receptor Identifies Effector CD8 T Cells That Give Rise to Long-Lived Memory Cells,” Nat Immunol 4:1191-8 (2003); Hendriks et al., “CD27 is Required for Generation and Long-Term Maintenance of T Cell Immunity,” Nat Immunol 1:433-40 (2000)). However, the effect of the migratory pattern of effector CD8+ T cells on their eventual fate is less well understood.

Migration to sites of infection is a primary requirement for the effective clearance of pathogens, but there is evidence to suggest that primed T cells also migrate to a variety of other peripheral lymphoid and non-lymphoid tissues (Masopust et al., “Preferential Localization of Effector Memory Cells in Nonlymphoid Tissue,” Science 291:2413-7 (2001); Marshall et al., “Measuring the Diaspora for Virus-Specific CD8+ T Cells,” Proc Natl Acad Sci USA 98:6313-8 (2001); Reinhardt et al., “Visualizing the Generation of Memory CD4 T Cells in the Whole Body,” Nature 410:101-5 (2001)). Amongst the peripheral tissues, the liver is a preferential site for the accumulation and disposal of CD8+ T cells at the end of a systemic immune response (Huang et al., “The Liver Eliminates T Cells Undergoing Antigen-Triggered Apoptosis in vivo,” Immunity 1:741-9 (1994)).

Activated CD8+ T cells, primed in response to an antigenic challenge, enter the blood and circulate widely through the tissues. These T cells undergo diverse fates. A subset of the cells undergoes apoptosis while others enter the memory pool. Among the cells that undergo apoptosis, an unusually large proportion are trapped in the liver due to the expression of Intercellular Adhesion Molecule-1 (ICAM-1) and Vascular Cell Adhesion Molecule-1 (VCAM-1) on hepatic sinusoidal endothelium (John et al., “Passive and Active Mechanisms Trap Activated CD8+ T Cells in the Liver,” J Immunol 172:5222 (2004)). Such trapping and intrahepatic apoptosis of activated CD8+ T cells is seen in mouse models driven by antigenic peptide, in Simian Immunodeficiency Virus infection, and in influenza infection (Belz et al., “Characteristics of Virus-Specific CD8+ T Cells in the Liver During the Control and Resolution Phases of Influenza Pneumonia,” Proc Natl Acad Sci USA 95:13812 (1998); Mehal et al., “Selective Retention of Activated CD8+ T Cells by the Normal Liver,” J Immunol 163:3202 (1999); and Crispe et al., “The Liver as a Site of T-Cell Apoptosis: Graveyard, or Killing Field? Immunol Rev. 174:47 (2000)), suggesting that the liver plays an important part in the elimination of the activated CD8+ T cells. It would thus be valuable to understand why the liver is the preferred site for such large-scale migration and destruction of the activated CD8+ T cells.

The unique immunological environment in the liver has been attributed to its close connection to the gut. The liver is exposed to microbial products synthesized by the commensal intestinal flora, a major component of which is endotoxin (lipopolysaccharide, LPS) from gram-negative bacteria (Nolan et al., “The Role of Endotoxin in Liver Injury,” Gastroenterology 69:1346 (1975); and Knolle et al., “Neighborhood Politics: The Immunoregulatory Function of Organ-Resident Liver Endothelial Cells,” Trends Immunol 22:432 (2001)). These microbial products are absorbed from the gut and transported via the portal vein to the liver. The portal venous blood entering the liver contains LPS at concentrations ranging from 100 pg/ml to 1 ng/ml, while virtually no LPS is detected in the hepatic venous blood that drains into the systemic circulation (Lumsden et al., “Endotoxin Levels Measured by a Chromogenic Assay in Portal, Hepatic and Peripheral Venous Blood in Patients with Cirrhosis,” Hepatology 8:232 (1988); and Freudenberg et al., “Time Course of Cellular Distribution of Endotoxin in Liver, Lungs and Kidneys of Rats,” Br J Exp Pathol 63:55 (1982)). This supports the idea that the liver is a local sink for LPS and the main site for its clearance (Nolan et al., “The Role of Endotoxin in Liver Injury,” Gastroenterology 69:1346 (1975); and Knolle et al., “Neighborhood Politics: The Immunoregulatory Function of Organ-Resident Liver Endothelial Cells,” Trends Immunol 22:432 (2001)). In the liver, Kupffer cells and liver sinusoidal endothelial cells (LSECs) are the main scavengers for LPS, although hepatocytes also take it up (Bikhazi et al., “Kinetics of Lipopolysaccharide Clearance by Kupffer and Parenchyma Cells in Perfused Rat Liver,” Comp Biochem Physiol C Toxicol Pharmacol 129:339 (2001); and Mimura et al., “Role of Hepatocytes in Direct Clearance of Lipopolysaccharide in Rats,” Gastroenterology 109:1969 (1995)).

Bacterial and viral molecules that contain conserved structural motifs (termed pathogen-associated molecular patterns, or PAMPs) engage pattern recognition receptors, many of which belong to the Toll-like receptor (TLR) family (Janeway et al., “Introduction: The Role of Innate Immunity in the Adaptive Immune Response,” Semin Immunol 10:349 (1998)). Toll-like receptors are the mammalian homologues of the Drosophila Toll protein, which is vital for morphogenesis in fruit flies but was surprisingly also found to be responsible for the resistance of the flies to fungal infections (Lemaitre et al., “The Dorsoventral Regulatory Gene Cassette Spatzle/Toll/Cactus Controls the Potent Antifungal Response in Drosophila Adults,”Cell 86:973 (1996)). Since the initial identification of TLR-4 (Medzhitov et al., “A Human Homologue of the Drosophila Toll Protein Signals Activation of Adaptive Immunity,” Nature 388:394 (1997)) and its co-localization with the receptor for LPS, ten TLRs have been identified in mammals, each of which recognizes distinct molecular patterns associated with different groups of pathogens (Iwasaki et al., “Toll-Like Receptor Control of the Adaptive Immune Responses,” Nat Immunol 5:987 (2004)). TLR-2 and TLR-4 are the two main components in the responsiveness to bacterial products and TLR-4 is essential for LPS mediated signaling (Takeda et al., “Toll-Like Receptors,” Annu Rev Immunol 21:335 (2003); and Poltorak et al., “Defective LPS Signaling in C3H/HeJ and C57BL/lOScCr Mice: Mutations in T1r4 Gene,” Science 282:2085 (1998)). Different cell populations in the liver, including Kupffer cells, LSECs, hepatocytes and hepatic stellate cells have been shown to express TLR-4 (Liu et al., “Role of Toll-Like Receptors in Changes in Gene Expression and NF-Kappa B Activation in Mouse Hepatocytes Stimulated with Lipopolysaccharide,” Infect Immun 70:3433 (2002); and Paik et al., “Toll-Like Receptor 4 Mediates Inflammatory Signaling by Bacterial Lipopolysaccharide in Human Hepatic Stellate Cells. Increase in Adhesion Molecules,” Hepatology 37:1043 (2003)) and can respond to exogenous LPS (Paik et al., “Toll-Like Receptor 4 Mediates Inflammatory Signaling by Bacterial Lipopolysaccharide in Human Hepatic Stellate Cells. Increase in Adhesion Molecules,” Hepatology 37:1043 (2003); and Kopydlowski et al., “Regulation of Macrophage Chemokine Expression by Lipopolysaccharide in vitro and in vivo,” J Immunol 1 63:1537 (1999)). The bacterial ligands recognized by TLRs are not unique to pathogens, but are also produced by the commensal microorganisms. Whether the cells of the liver can respond to the basal physiological levels of the commensal-derived products and, if so, the consequences of these responses both remain to be determined.

Mice that lack a constant source of LPS entering their liver (germ-free mice) have reduced expression of the adhesion molecule ICAM-1 in their livers and a normal level of expression can be restored by the intragastric inoculation of cecal micro flora from normal mice (Komatsu et al., “Enteric Micro Flora Contribute to Constitutive ICAM-1 Expression on Vascular Endothelial Cells,” Am J Physiol Gastrointest Liver Physiol 279:G186 (2000)). Both germ-free mice and TLR-4 deficient mice (Kiyono et al., “Lack of Oral Tolerance in C3H/HeJ Mice,” J Exp Med 155:605 (1982)) show defective oral tolerance, while other studies show that the liver is involved in this process (Watanabe et al., “A Liver Tolerates a Portal Antigen by Generating CD1lc+ Cells, Which Select Fas Ligand+ Th2 Cells via Apoptosis,”Hepatology 38:403 (2003); and Yang et al., “Intestinal Venous Drainage Through the Liver is a Prerequisite for Oral Tolerance Induction,” J Pediatr Surg 29:1145 (1994)).

The best-understood function of TLR signaling is to activate the innate arm of the immune system, initiating host defense and promoting the priming of antigen-specific immunity (Takeda et al., “Toll-Like Receptors,” Annu Rev Immunol 21:335 (2003)). In the liver, it is difficult to understand how immune tolerance to harmless commensal bacteria is maintained despite the continuous exposure of the liver to TLR-2 and TLR-4 ligands. Work from other groups suggested the possibility that the response of LSECs and of Kupffer cells to LPS was unusual. While LPS causes Kupffer cells and LSEC to produce inflammatory cytokines, these are counterbalanced by the anti-inflammatory cytokines such as IL-10 and TGF-beta that are also released by these cells in response to LPS (Knolle et al., “Regulation of Endotoxin-Induced IL-6 Production in Liver Sinusoidal Endothelial Cells and Kupffer Cells by IL-10,” Clin Exp Immunol 107:555 (1997); and Knolle et al., “Human Kupffer Cells Secrete IL-10 in Response to Lipopolysaccharide (LPS) Challenge,” J Hepatol 22:226 (1995)). Physiological concentrations of endotoxin have also been shown to down-regulate T cell activation by antigen presenting LSECs (Knolle et al., “Endotoxin Down-Regulates T Cell Activation by Antigen-Presenting Liver Sinusoidal Endothelial Cells,” J Immunol 162:1401-7 (1999)). However, the details of how TLR-4 modulates the interaction between T cells and the liver, and how this might be manipulated to enhance immune response, remain unclear.

The present invention is directed to overcoming these and other deficiencies in the prior art.

### SUMMARY OF THE INVENTION

A first aspect of the present invention relates to a method of inhibiting intrahepatic CD8+ T cell deletion. The method involves providing a TLR-4 inhibitor and administering the inhibitor to a subject in an amount effective to inhibit intrahepatic CD8+ T cell deletion.

A second aspect of the present invention relates to a composition comprising a TLR-4 inhibitor and an immunogenic agent.

A third aspect of the present invention relates to a composition comprising a TLR-4 inhibitor and activated CD8+ T cells.

A fourth aspect of the present invention relates to a method of enhancing a secondary immune response in a subject. The method involves providing a composition according to the second aspect of the present invention or a combination of a TLR-4 inhibitor and an immunogenic agent, and administering the composition or the combination to a subject in an amount effective to activate a T cell response while inhibiting intrahepatic deletion of activated T cells. This method increases the survival of memory cells affording an enhanced secondary immune response to the immunogenic agent, T cell activating pathogen, or its equivalent.

A fifth aspect of the present invention relates to a method of enhancing a secondary immune response in an immuno-compromised subject. The method involves providing a composition according to the third aspect of the present invention or a combination of a TLR-4 inhibitor and activated CD8+ T cells, and administering the composition or the combination to an immuno-compromised subject in an amount effective to promote survival of memory cells. This method affords an enhanced secondary immune response to an immunogenic agent, T cell activating pathogen, or its equivalent.

A sixth aspect of the present invention relates to a method of enhancing a secondary immune response in a subject. The method involves administering to a subject an amount of a TLR-4 inhibitor that is effective to promote the survival of memory cells. This affords an enhanced secondary immune response to an immunogenic agent, T cell activating pathogen, or its equivalent.

The present invention provides a unique technique for enhancing immune cell memory by inhibiting TLR-4 activity in the liver. In the accompanying examples, CD8+ T cells were activated either by antigen specific T cell receptor (TCR) ligation, or using cells expressing a superantigen, and the localization of the responding CD8+ T cells in TLR-4 non-responsive mice was determined. The examples show that TLR-4 plays an important part in the ability of the liver to trap activated CD8+ T cells. The examples further demonstrate, using wild type and TLR-4 deficient mice (which received an adoptive transfer of OT1 CD8+ T cells that were primed using wild type in vitro antigen-loaded antigen-presenting cells), that TLR-4 compromises trapping in the liver. This was confirmed by orthotopic liver transfer studies. Therefore, by blocking or interfering with (inhibiting) intrahepatic CD8+ T cell deletion, it is possible to afford enhanced secondary immune responses, both in normal, healthy individuals and, more particularly, in individuals who may be immuno-compromised. The present invention affords an important tool in vaccination.

### BRIEF DESCRIPTION OF THE DRAWINGS

FIGS. 1A-C show that TLR-4 influences the recirculation of activated CD8+ T cells between the liver and the blood. The expression of the activation markers CD44, CD69, CD62L and CD25 on the activated OT1 T cells (CD45.1×CD45.2) and naïve CD8+ T cells (CD45.1) before injection is shown in FIG. 1A. The percentage of CD45.1/CD45.2 double positive (activated) and CD45.1 single positive (naïve) OT1 cells amongst the total CD45.1 CD8+ cells from the liver, spleen, peripheral lymph nodes (marked LN) and the peripheral blood (PBMC) of wt and TLR-4−/− mice is shown in FIG. 1B. FIG. 1C shows an average (n=5) of the ratio of activated to naïve OT1 cells in the spleen (gray bars), lymph nodes (dotted bars), liver (black bars) and peripheral blood (hatched bars) of the wt and TLR-4 deficient mice. The 0 hr time point (open bar) indicates the ratio of the cells prior to transfer.

FIGS. 2A-C show that TLR-4−/− mice retain fewer activated OT1 cells in their livers compared to wildtype mice in an in situ immune response. FIG. 2A shows the percentage of OT1 cells (CD45.1 Valpha2) cells on day 5 in the spleen, lymph nodes and liver of WT or TLR-4−/−mice which received OT1 cells and were activated with splenic dendritic cells pulsed with SIINFEKL peptide. The average OT1 percentage (FIG. 2B) and cell numbers (FIG. 2C) in the spleen (hatched bars), lymph nodes (empty bars) and liver (filled bars) of WT or TLR-4−/− mice 3 (top panels) and 5 days (bottom panels) after immunization is also depicted in the figure.

FIG. 3 shows that the activation of adoptively transferred OT1 T cells is comparable between WT and TLR-4−/− mice. The data show the percentage of OT1 cells (CD45.1+Valpha2+) in the spleen, lymph nodes and liver of WT and TLR-4−/− mice 3 days after they were given either unpulsed APCs or SIINFEKL peptide (SEQ ID NO:1) pulsed APCs. The figure also shows the down-regulation of CD62L and the up-regulation of CD44 upon activation of the OT1 cells in the WT and TLR-4−/− mice.

FIG. 4 shows that wildtype and TLR-4 deficient mice are comparable in their ability to proliferate and synthesize IFN-gamma. The data show the dilution of CFSE as a function of IFN-gamma synthesis on the gated CD45.1+Valpha2+ cells (OT1 cells) from spleen, lymph nodes and liver of WT or TLR-4−/− mice, 3 days after they were given unpulsed or peptide pulsed APCs. The cells were restimulated in culture for 6 hours with or without the specific antigenic peptide, SIINFEKL (SEQ ID NO:1). The data are representative of three separate experiments with 3 mice per group in each experiment.

FIGS. 5A-B show the OT1 cells activated in both WT and TLR-4 deficient mice are equally cytotoxic: FIG. 5A shows the CFSE levels on the unpulsed and SIINFEKL peptide pulsed targets prior to transfer. FIG. 5A also shows the percentage of the OT1 cells (CD45.1) and the two different target cell populations (CFSE^{high} and CFSE^{low}) in the lymph nodes of WT and TLR-4−/− mice. FIG. 5B shows an average of the percentage target cell lyses in the lymph nodes (gray bars), spleen (open bars) and liver (black bars) of WT and TLR-4−/− mice (n=5 per group). The percentage target cell lyses was calculated using the formula {1−(% of peptide pulsed target/% of unpulsed targets)}×100.

FIGS. 6A-B show that TLR-4 mutant mice accumulate fewer activated CD8+ T cells in their livers compared to control mice. FIG. 6A shows the percentage of Vbeta6 CD8+ T cells in the lymph nodes and livers of TLR-4 mutant (C3H/HeJ) and control mice (C3H/HeOuJ) mice before (day 0) and 8 days after exposure of the antigen. In FIG. 6B the Vbeta6 CD8+ T cell percentage is plotted as a ratio of the total CD8+ T cell percentage in the lymph nodes (top panel) and liver (bottom panel) of C3H/HeJ (open bars) and C3H/HeOuJ (hatched bars) mice at different time points (days 2, 4, 6, 8, and 15) after injection of the AKR/J splenocytes. N=6 at each time point for each of the experimental groups.

FIGS. 7A-B show that TLR-4 deficient mice possess a higher frequency of CD8+ memory precursors compared to WT mice. FIG. 7A shows the percentage of OT1 T cells (CD45.1+CD8+) in the peripheral blood of either WT (closed symbols) or TLR-4−/− (open symbols) at various points (days 0, 3, 5, 12, 20 and 35) after primary immunization with peptide pulsed APCs. The percentage of the OT1 cells in the spleen, liver, bone marrow and lymph nodes of the WT (black bars) or TLR-4−/− mice (open bars) 6 weeks after primary immunization with peptide is represented in Panel B of the figure. N>12 for each of the groups. The differences in FIG. 7B between the WT and TLR-4−/− mice were found to be significant (P=0.025) additively in the four tissues, as tested by a 2×4 factorial ANOVA.

FIG. 8 shows that CD8+ memory T cell precursors primed in wildtype and TLR-4 deficient hosts are functionally and phenotypically identical. The expression of the activation markers CD62L, CD44 and CD127 on the OT1 cells in WT (left panel) or TLR-4 deficient mice (right panel), six weeks after primary immunization with peptide, is shown in FIG. 8. Also shown in FIG. 8 is the production of IFN-gamma by the OT1 cells after 6 hours of re-stimulation with/without SIINFEKL peptide (SEQ ID NO:1) in culture. The data are representative of at least 10 mice in each group.

FIGS. 9A-B show that T cells primed in TLR-4 deficient mice show better recall responses 6 weeks after immunization. Both the percentage (FIG. 9A) and numbers (FIG. 9B) of OT1 TCR transgenic CD8+ T cells were measured in the liver, lymph nodes and spleens of WT or TLR-4 deficient mice six weeks after primary immunization (1°) with SIINFEKL peptide (SEQ ID NO:1) pulsed APCs. In the secondary challenge (2°) the mice either received PBS or SIINFEKL peptide and all the responses were measured on day 3 after secondary challenge. The data shown is an average of 11 mice in each of the groups. The significance values were obtained using the student t test (unpaired, 2 tailed).

FIGS. 10A-B show that secondary clonal expansion is controlled by host TLR-4 expression. Memory cells generated in the WT or TLR-4 deficient mice, transferred in equal numbers into WT mice, expand to the same extent. However they expand more when transferred into a TLR-4 deficient host. FIG. 10A shows the percentage of the OT1 memory cells generated in either WT or TLR-4−/− mice that were retransferred into either WT or TLR-4 deficient mice. The responses shown are before (day 0) and 3 days after challenge with SIINFEKL peptide (SEQ ID NO:1) in saline ((day 3). The dilution of CFSE by the OT1 cells (CD45.1+) in the peripheral blood is also shown in FIG. 10A. FIG. 10B shows the average percentage of OT1 memory cells that have expanded in the spleen, lymph nodes, peripheral blood and liver of WT or TLR-4 deficient hosts 3 days after challenge with peptide (n=6 in each group). The significance values were obtained using the student's t test.

FIGS. 11A-B show that wildtype mice transplanted with TLR-4 deficient livers display the same phenotype as that seen in intact TLR-4 deficient mice. The percentage (FIG. 11A) and cell numbers (FIG. 11B) of OT1 TCR transgenic cells in the liver, lymph nodes, and spleens of WT mice that were transplanted with WT livers (WT->WT) or WT mice that were transplanted with TLR-4−/− livers (TLR-4->WT) are shown. The primary immunization was with peptide pulsed APCs, and mice were re-challenged with either PBS or SIINFEKL peptide (SEQ ID NO:1) six weeks after primary immunization. The data shown are an average of 6 mice per group. The significance values were obtained by a 2×3 factorial ANOVA (VassarStats).

### DETAILED DESCRIPTION OF THE INVENTION

The present invention relates generally to methods and compositions for inhibiting intrahepatic activated T cell deletion. Various methods and compositions can be used to enhance active immune responses in subjects, while still other methods and compositions can be used to enhance the efficacy of passive immunotherapy procedures in subjects, particularly immunocompromised subjects.

One embodiment of the present invention relates to a method of inhibiting intrahepatic CD8+ T cell deletion by providing a TLR-4 inhibitor, and then administering the inhibitor to a subject in an amount effective to inhibit intrahepatic CD8+ T cell deletion. It is preferable that the inhibitors are administered to form a transient blockade of TLR-4 function, thereby neutralizing the effect of TLR-4 on intrahepatic CD8+ T cell deletion while maintaining a desirable CD8+ T cell immune response. Basically, it is desirable to inhibit T cell deletion during the period of time soon after activation, while the T cells remain circulating (i.e., before the cells return to a resting state). This will increase the population of resting T cells (memory cells), and thereby enhance secondary immune responses to an immunogenic agent, T cell activating pathogen, or equivalents thereof.

The subject can be any mammal including, without limitation, a human, a non-human primate, a mouse, a rat, a guinea pig, a rabbit, a cat, a dog, a horse, a cow, a sheep, a goat, a pig, etc. According to one embodiment, the subject is not immunocompromised and, therefore, is expected to mount a typical immune response following vaccination. According to another embodiment, the subject is immunocompromised. Depending upon the severity of the subject's immune deficiency, either traditional vaccinations can be used or passive immunization procedures can be used, both of which will be augmented by the methods and compositions of the present invention. As an issue of safety, the TLR-4 inhibitor should not be administered to a subject being treated for an active case of sepsis, as the infection implicates TLR-4 recognition and should not be inhibited.

The TLR-4 inhibitor can be an anti-TLR-4 antibody, a nucleic acid expressing antisense TLR-4 RNA or siRNA, a nucleic acid encoding a ribozyme that cleaves TLR-4 mRNA, an antisense TLR-4 oligodeoxynucleotide, a nucleic acid aptamer specific for TLR-4 or its mRNA, a TLR-4 polypeptide sequence that corresponds to at least a portion of the receptor and binds to a TLR-4 ligand during TLR-4 signal transduction event, a non-TLR-4 protein or polypeptide that inhibits TLR-4 activity, a small molecule inhibitor of TLR-4 activity, or an inhibitory ligand that is a variant of the natural ligand of TLR-4, namely bacterial lipopolysaccharide. Regardless of the type of inhibitor employed, the TLR-4 inhibitor is then administered to achieve transient blockade of TLR-4 function, thereby neutralizing or at least partially inhibiting the effect of TLR-4 on intrahepatic CD8+ T cell deletion. This reduces the extent of CD8+ T cell contraction, and concomitantly enhances the population of resting T cells. As a consequence, secondary immune responses will be enhanced significantly.

Suitable polypeptide fragments of the TLR-4 may include at least a portion of the receptor sequence that binds to a TLR-4 ligand, are preferably short polypeptides from about 10 to 100 or 10 to 50 aa in length (or smaller), which contain the TLR-4 ligand binding domain. The peptide fragments can also be part of an N-terminal or C-terminal fusion protein. The full length sequence of various human TLR-4 isoforms are known (see Genbank Accession Nos. NP_{—}612564 (isoform A), NP_{—}612566 (isoform B), NP_{—}003257 (isoform C), and NP_{—}612567 (isoform D), each of which is hereby incorporated by reference in its entirety). Sequences for other mammalian TLR-4 homologs are also known, including those of mouse, rat, orangutan, etc.

Non-TLR-4 protein or polypeptide inhibitors of TLR-4 have also been identified in the literature, and these can be used. Two such inhibitors are identified in Yang et al., “Novel TLR-4 Antagonizing Peptides Inhibit LPS-Induced Release of Inflammatory Mediators by Monocytes,” Biochem. Biophys. Res. Commun. 329(3):846-54 (2005), which is hereby incorporated by reference in its entirety; and chemokine receptor 4 and its ligand have also been shown to be effective (Kishore et al., “Selective Suppression of Toll-like Receptor 4 Activation by Chemokine Receptor 4,” FEBS Lett. 579(3):699-704 (2005), which is hereby incorporated by reference in its entirety).

The anti-TLR-4 antibodies can be monoclonal or polyclonal, and can be raised and isolated according to known procedures. Polyclonal antiserum can be rendered substantially monospecific using known procedures. Monoclonal antibodies can also be active fragments thereof, including without limitation, Fab fragments, F(ab′)₂ fragments, and Fv fragments. These monoclonal antibodies (and fragments or variants thereof) can be humanized using known procedures. The anti-TLR-4 antibodies can be administered in any suitable pharmaceutical composition, but preferably those utilized for delivery of isolated antibodies, e.g., for passive immunity or other forms of antibody therapy.

Exemplary TLR-4 antagonists include, without limitation, Rhodobacter sphaeroides lipid A, which is a specific antagonist of TLR-4; E5564 (also known as compound 1287, SGEA, and Eriforan) (Mullarkey et al., “Inhibition of Endotoxin Response by E5564, a Novel Toll-like Receptor 4-directed Endotoxin Antagonist,” J. Pharmacol. Exp. Ther. 304(3):1093-1102 (2003); Hawkins et al., “Inhibition of Endotoxin Response by Synthetic TLR4 Antagonists,” Curr Top Med. Chem. 4(11):1147-1171 (2004); U.S. Pat. No. 5,681,824 to Christ et al., each of which is hereby incorporated by reference in its entirety); TAK-242 (Ii et al., “A Novel Cyclohexene Derivative, ethyl (6R)-6-[N-(2-Chloro-4-fluorophenyl)sulfamoyl]cyclohex-1-ene-1-carboxylate (TAK-242), Selectively Inhibits Toll-like Receptor 4-mediated Cytokine Production Through Suppression of Intracellular Signaling,” Mol Pharmacol. 69(4):1288-95 (2006), which is hereby incorporated by reference in its entirety); the endogenous TLR-4 inhibitor RP105 (Divanovic et al., “Inhibition of TLR-4/MD-2 signaling by RP 105/MD-1,” J Endotoxin Res. 11(6):363-368 (2005), which is hereby incorporated by reference in its entirety); the lipid A-mimetic CRX-526 (Fort et al., “A Synthetic TLR4 Antagonist Has Anti-Inflammatory Effects in Two Murine Models of Inflammatory Bowel Disease,” J Immunol 174:6416-6423 (2005), which is hereby incorporated by reference in its entirety); CyP, a natural LPS mimetic derived from the cyanobacterium Oscillatoria planktothrix FP1 (Macagno et al., “A Cyanobacterial LPS Antagonist Prevents Endotoxin Shock and Blocks Sustained TLR4 Stimulation Required for Cytokine Expression,” J. Exp. Med. 203(6):1481-1492 (2006), which is hereby incorporated by reference in its entirety; a phenol/water extract from T. socranskli subsp. socranskii (TSS-P) (Lee et al., “Phenol/water Extract of Treponema socranskii subsp. socranskii as an Antagonist of Toll-like Receptor 4 Signaling,” Microbiol. 152(2):535-46 (2006), which is hereby incorporated by reference in its entirety); CLR proteins such as Monarch-1 (Williams et al., “The CATERPILLAR Protein Monarch-1 Is an Antagonist of Toll-like Receptor-, Tumor Necrosis Factor alpha-, and Mycobacterium tuberculosis-induced pro-inflammatory signals,” J. Biol. Chem. 280(48):39914-39924 (2005), which is hereby incorporated by reference in its entirety); and small molecule TLR-4/TLR-2 dual antagonists, such as ER811243, ER811211, and ER811232 (U.S. Patent Application Publ. No. 20050113345 to Chow et al., which is hereby incorporated by reference in its entirety).

In the aspect of the present invention in which down-regulation of TLR-4 expression is desired, the method may involve an RNA-based form of gene-silencing known as RNA-interference (RNAi) (also known more recently as siRNA for short, interfering RNAs). Suitable TLR-4 mRNA target sequences can be, but are not limited to, those from human, mouse, and rat (see, e.g., GenBank Accession Nos. NM003266, NM021297, NM019178, each of which is hereby incorporated by reference in its entirety). Numerous reports have been published on critical advances in the understanding of the biochemistry and genetics of both gene silencing and RNAi (Matzke et al., “RNA-Based Silencing Strategies in Plants,” Curr. Opin. Genet. Dev., 11(2):221-227 (2001), which is hereby incorporated by reference in its entirety). In RNAi, the introduction of double stranded RNA (dsRNA) into animal or plant cells leads to the destruction of the endogenous, homologous mRNA, phenocopying a null mutant for that specific gene. In both post-transcriptional gene silencing and RNAi, the dsRNA is processed to short interfering molecules of 21-, 22- or 23-nucleotide RNAs (siRNA) by a putative RNAaseIII-like enzyme (Tuschl, “RNA Interference and Small Interfering RNAs,” Chembiochem 2: 239-245 (2001); Zamore et al., “RNAi: Double Stranded RNA Directs the ATP-Dependent Cleavage of mRNA at 21 to 23 Nucleotide Intervals,” Cell 101, 25-3, (2000), which are hereby incorporated by reference in their entirety). The endogenously generated siRNAs mediate and direct the specific degradation of the target mRNA. In the case of RNAi, the cleavage site in the mRNA molecule targeted for degradation is located near the center of the region covered by the siRNA (Elbashir et al., “RNA Interference is Mediated by 21- and 22-Nucleotide RNAs,” Gene Dev. 15(2):188-200 (2001), which is hereby incorporated by reference in its entirety).

In one aspect, dsRNA for the nucleic acid molecule of the present invention can be generated by transcription in vivo. This involves modifying the nucleic acid molecule of the present invention for the production of dsRNA, inserting the modified nucleic acid molecule into a suitable expression vector having the appropriate 5′ and 3′ regulatory nucleotide sequences operably linked for transcription and translation, and introducing the expression vector having the modified nucleic acid molecule into a suitable host cell or subject. In another aspect of the present invention, complementary sense and antisense RNAs derived from a substantial portion of the coding region of the nucleic acid molecule of the present invention are synthesized in vitro. (Fire et al., “Specific Interference by Ingested dsRNA,” Nature 391:806-811 (1998); Montgomery et al, “RNA as a Target of Double-Stranded RNA-Mediated Genetic Interference in Caenorhabditis elegans,” Proc. Natl Acad Sci USA 95: 15502-15507; Tabara et al., “RNAi in C. elegans: Soaking in the Genome Sequence,” Science 282:430-431 (1998), which are hereby incorporated by reference in their entirety). The resulting sense and antisense RNAs are annealed in an injection buffer, and dsRNA is administered to the subject using any method of administration described herein, infra.

In the aspect of the present invention where the TLR-4 inhibitor is a nucleic acid encoding a ribozyme that cleaves TLR-4 mRNA, ribozymes may be synthesized using methods commonly known to those skilled in the art (see Ohmichi et al., “Development of Ribozyme Synthesis System Using a Rolling-Synchronization: Effect of Template DNA Secondary Structure on Recognition of RNA Polymerase,” Nucleic Acids Res. Suppl., 1:37-38 (2001); Bellon et al., “Post-synthetically Ligated Ribozymes: An Alternative Approach to Iterative Solid-Phase Synthesis,” Bioconjug. Chem. 8:204-12 (1997); Chow et al., “Synthesis and Purification of a Hammerhead Ribozyme and a Fluorescein-Labeled RNA Substrate. A Biochemistry Laboratory: Part 1,” J. Chem. Educ. 76:648 (1999), which are hereby incorporated by reference in their entirety).

In another aspect, the inhibitor of TLR-4 can be a nucleic acid aptamer (DNA or RNA). Aptamers can be selected from libraries screened for their ability to bind TLR-4 and perturb its activity. The techniques for selecting aptamers against specific targets, forming multivalent aptamers based upon the selected individual aptamers, and their use have been described. See, e.g., U.S. Pat. No. 6,458,559 to Shi et al., and U.S. Patent Application Publ. No. 20040053310 to Shi et al., each of which is hereby incorporated by reference in its entirety.

The one or more inhibitors of the present invention can be administered orally, topically, transdermally, parenterally, subcutaneously, intravenously (e.g., hepatic vein), intramuscularly, intraperitoneally, intracavitary, by intravesical instillation, intranasally, intraocularly, intraarterially, intralesionally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The administration of the TLR-4 inhibitor can be performed repeatedly during the normal, activated T cell expansion and contraction phases, particularly from the first day of exposure to an antigen up to about 60 days, more preferably between days 0-30 or 0-15 post-exposure. The repeat administrations of TLR-4 inhibitors can be up to several times daily or less frequent, depending on the half-life of the particular inhibitor.

The inhibitors of the present invention may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these active compounds may be incorporated with excipients and used in the form of tablets, capsules, elixirs, suspensions, syrups, and the like. Such compositions and preparations should contain at least 0.1% of active compound. The percentage of the compound in these compositions may, of course, be varied and may conveniently be between about 2% to about 60% of the weight of the unit. The amount of active compound in such therapeutically useful compositions is such that a suitable dosage will be obtained.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to active ingredient, sucrose as a sweetening agent, methyl and propylparabens as preservatives, a dye, and flavoring such as cherry or orange flavor.

These inhibitors may also be administered parenterally. Solutions or suspensions of these active compounds can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The inhibitors of the present invention may also be administered directly to the airways in the form of an aerosol. For use as aerosols, the inhibitors of the present invention in solution or suspension may be packaged in a pressurized aerosol container together with suitable propellants, for example, hydrocarbon propellants like propane, butane, or isobutane with conventional adjuvants. The materials of the present invention also may be administered in a non-pressurized form such as in a nebulizer or atomizer.

Persons of skill in the art are readily able to test and assess optimal dosage schedules based on the balance of efficacy and any undesirable side effects. The optimal dosage of each type of inhibitor will vary, of course, and the minimal effective dose will be administered for therapeutic regimen.

The TLR-4 inhibitors can be administered alone, in combination with an immunogenic agent or activated CD8+ T cells (i.e., as distinct doses), or in the form of a single composition containing the TLR-4 inhibitor and one or both of the immunogenic agent and the activated CD8+ T cells.

According to one embodiment, a composition includes a TLR-4 inhibitor and an immunogenic agent. The inhibitor can be any one or more of the TLR-4 inhibitors described above. The immunogenic agent can be a polypeptide comprising an epitope of a T cell activating pathogen where the pathogen is a bacterium, a virion, a parasite or an immunogenic cancer. Alternatively, the immunogenic agent can be a pathogen that has been disabled, or a pathogen mimic (such as a virus-like particle). Exemplary T cell activating pathogen include, without limitation, Listeria monocytogenes, Leishmania leishmaniasis, Chlamydia trachomatis, Mycobacterium tuberculosis, Influenza sp., Trypanosoma cruzi, Lentivirus sp. (e.g., HIV) or a Hepacivirus sp.

The composition can also include a pharmaceutically acceptable carrier, where the composition is in the form of a vaccine, and an adjuvant may also be present. Any suitable adjuvant can be used, but preferably the adjuvant does not function solely via TLR-4. Exemplary adjuvants of this type include, without limitation, an inflammatory cytokine.

In addition, the composition can be present in a delivery vehicle designed for administration. The delivery vehicle can be any suitable delivery vehicle. Exemplary delivery vehicles include, without limitation, single-use injection devices; polymeric delivery vehicles, implantable or otherwise; polyketal nanoparticles; liposomal particles; and a gene therapy vector.

According to another embodiment, a composition includes activated CD8+ T cells and a TLR-4 inhibitor. The inhibitor can be any of the TLR-4 inhibitors as described above. The activated CD8+ T cells can be isolated from an individual exposed to a systemic immunogenic challenge where the individual can be a mammal, including those described above in connection with the present invention. Preferably, the individual is the same species as the subject intended to receive the composition. Isolation of activated CD8+ T cells can be accomplished by methods commonly known to persons of skill in the art (see Zhou et al., “Diverse CD8+ T-cell Responses to Renal Cell Carcinoma Antigens in Patients Treated with an Autologous Granulocyte-macrophage Colony-stimulating Factor Gene-transduced Renal Tumor Cell Vaccine”, Cancer Res. 65:1079-88 (2005); Rufer et al., “Methods for the ex vivo Characterization of Human CD8+ T Subsets Based on Gene Expression and Replicative History Analysis,” Methods Mol. Med. 109:265-284 (2005), which are hereby incorporated by reference in their entirety). The composition can further comprise a pharmaceutically acceptable carrier or may be present in a delivery vehicle as described above. Administration can be achieved using the above-described routes, but preferably via a systemic delivery route (e.g. intravenous or intraarterial).

Another aspect of the present invention relates to a method of enhancing a secondary immune response in a subject. The method involves providing a composition that includes a TLR-4 inhibitor and an immunogenic agent or a combination of the TLR-4 inhibitor and the immunogenic agent (i.e., as distinct compositions), and administering the composition or the combination to a subject in an amount effective to activate a T cell response while inhibiting intrahepatic deletion of activated T cells. This method increases the survival of memory cells, affording an enhanced secondary immune response to the immunogenic agent, T cell activating pathogen, or its equivalent.

The TLR-4 inhibitor and the immunogenic agent can be any of those described above in connection with the present invention.

The method can involve repeat administrations of effective amounts of the composition, or either one or both of the TLR-4 inhibitor and the immunogenic agent, after the initial administration. Thus, the TLR-4 inhibitor can be administered more frequently than the immunogenic agent or vice versa. The delay between repeat administrations can be adjusted to optimize results, but preferably the repeat administrations are carried out during the expansion and contraction phases as described above.

Another aspect of the present invention relates a method of enhancing a secondary immune response in an immuno-compromised subject. The method involves providing a composition that includes activated CD8+ T cells and a TLR-4 inhibitor or a combination of the activated CD8+ T cells and the TLR-4 inhibitor (i.e., as distinct compositions), and administering the composition or the combination to an immuno-compromised subject in an amount effective to promote survival of effector and memory T cells. This method affords an enhanced secondary immune response to an immunogenic agent, T cell activating pathogen, or its equivalent (i.e., against which the CD8+ T cells were activated).

The method can also involve repeat administrations of effective amounts of the composition, or either one or both of the TLR-4 inhibitor and the activated CD8+ T cells, after the initial administration. The method can also involve the administration of effective amounts of a TLR-4 inhibitor following a delay after administration of the composition or the combination. Thus, the TLR-4 inhibitor can be administered more frequently than the activated CD8+ T cells, or vice versa. The delay between repeat administrations of the TLR-4 inhibitor are carried out during the contraction phase, substantially as described above.

Another aspect of the present invention relates to a method of enhancing a secondary immune response in a subject. The method involves administering to a subject an amount of a TLR-4 inhibitor that is effective to promote the survival of memory cells. This affords an enhanced secondary immune response to an immunogenic agent, T cell activating pathogen, or its equivalent. The TLR-4 inhibitor can also be administered if and when a patient is known to have been exposed (or is likely to have been exposed) to a particular pathogen.

In one embodiment, a vaccine that includes an immunogenic agent can be administered to the subject. The vaccine may be administered prior to, contemporaneously with, or subsequently to, administration of the TLR-4 inhibitor. The method can involve repeat administrations of effective amounts of the TLR-4 inhibitor as described above, and if multiple boosts of the vaccine are provided, then administration of the TLR-4 inhibitor can be carried out during each expansion and contraction phase during the boost regimen.

The extensive literature on TLRs emphasizes their role in augmenting and initiating innate immune responses. Thus, TLRs are involved in the maturation of specialized antigen presenting cells such as dendritic cells, the induction of co-stimulatory molecules, production of cytokines and chemokines by the cells of the innate immune system, and in the resistance of DC to regulatory T cells (Iwasaki et al., “Toll-Like Receptor Control of the Adaptive Immune Responses,” Nat Immunol 5:987 (2004); and Takeda et al., “Toll-Like Receptors,” Annu Rev Immunol 21:335 (2003), each of which is hereby incorporated by reference in its entirety). However, in recent years several other aspects of TLR biology have emerged. In the liver, antigen presentation is strongly influenced by LPS but in an unexpected way; endotoxin down-regulates T cell activation by LSECs and the CD4+ and CD8+ T cells that are activated by LSECs show a tolerant phenotype (Knolle et al., “Liver Sinusoidal Endothelial Cells can Prime Naive CD4+ T Cells in the Absence of IL-12 and Induce IL-4 Production in Primed CD4+ T Cells: Implications for Tolerance Induction in the Liver,” Gastroenterology 116:1428 (1999); and Limmer et al., “Efficient Presentation of Exogenous Antigen by Liver Endothelial Cells to CD8+ T Cells Results in Antigen-Specific T-Cell Tolerance,” Nat Med 6:1348 (2000), each of which is hereby incorporated by reference in their entirety). Thus, in this context, TLR engagement is immunosuppressive. Similarly, LPS acting on Kupffer cells and LSECs lead to the secretion of the immunosuppressive mediators such as IL-10 and TGF-beta (Knolle et al., “Control of Immune Responses by Scavenger Liver Endothelial Cells,” Swiss Med Wkly 133:501 (2003), which is hereby incorporated by reference in its entirety). More recently, the recognition of commensal-derived products by TLRs has been shown to play an important role in normal intestinal epithelial homeostasis (Rakoff-Nahoum et al., “Recognition of Commensal Microflora by TLRs is Required for Intestinal Homeostasis,” Cell 118:229 (2004), which is hereby incorporated by reference in its entirety). The present invention indicates a different function for TLR-4 under non-inflammatory conditions; TLR-4 ligands, possibly from the normal enteric flora, have a direct effect on the ability of the liver to trap activated CD8+ T cells.

As a consequence, the present invention affords an approach for supplementing secondary immune responses in individuals, whether they are immunocompromised or not. The present invention, therefore, is also expected to be useful for treatment of viral and fungal infections that spread through cell-to-cell interactions, e.g., influenza, malaria, CMV, HIV, etc., and in the treatment of viral infections and cancer by adoptive immunotherapy using CD8+ T cells.

### EXAMPLES

The following examples are provided to illustrate embodiments of the present invention but are by no means intended to limit its scope.

### Materials and Methods for Examples 1-5

Mice: TLR-4 deficient mice (C57BL/10 ScN), their WT counterparts (C57BL/10 SnJ), TLR-4 mutant mice (C3H/HeJ), their WT counterparts (C3H/HeOuJ), and the AKR/J strains of mice were purchased from the Jackson Laboratory (Bar Harbor, Me.) and housed in a specific pathogen-free environment in compliance with institutional guidelines for animal care. A colony of OT1 transgenic mice was maintained on a homozygous CD45.1 background and another colony was maintained on a heterozygous CD45.1 and CD45.2 background. A colony of OT1 transgenic mice (originally on a C57B16/J background) was extensively backcrossed with B6.SJL mice to obtain the CD45.1 homozygous OT1 transgenic mice. A second colony of OT1 transgenic mice was maintained on a CD45.1/CD45.2 heterozygous background by crossing CD45.1+/+OT1 transgenics with C57B1/6J (CD45.2+/+) mice.

CD8+ T cells for localization experiments: Lymphocytes were isolated from the spleen and peripheral lymph nodes of OT1 TCR transgenic mice, which were on a CD45.1 homozygous background. They were activated in vitro for 72 hours with 1 micromolar SIINFEKL (SEQ ID NO:1) peptide in the presence of spleen APC. This was used as a source of activated CD8+ T cells. Lymphocytes isolated from spleens and peripheral lymph nodes of OT1 TCR transgenic mice, on a CD45.1+/CD45.2+ heterozygous background, were used as a source of the naïve CD8+ T cells. Equal numbers of activated and naïve cells (10×10⁶ of each) were injected into either WT or TLR-4 deficient mice intravenously. The recipient mice were either WT (C57B1/10SnJ) or were TLR-4−/− (C57B1/10Scn), and were all on a CD45.2 background. Two hours later, the homing of the two different cell types to various compartments was analyzed. The activated, naïve and host cells were all distinguished from one another based on their expression of the allotypic markers, CD45.1, CD45.2 or both.

Isolation of splenic dendritic cells: Dendritic cells (DC) were enriched from the spleen using the technique established by Livingstone, “Isolation of CD4+ and CD8+ T-Cell Clones from Mice Immunized with Synthetic Peptides on Splenic Dendritic Cells,” Methods 9:422 (1996), which is hereby incorporated by reference in its entirety. Briefly, spleens were digested in an enzyme cocktail containing 2.4 mg/ml collagenase IV (Sigma, St. Louis, Mo.) and 1 mg/ml DNAse (Sigma) for 30 minutes at 37° C. The spleen cell digest was made into a single cell suspension with a syringe and needle followed by 2 washes with Hanks Balanced Salt Solution (HBSS). The cell pellet was then resuspended in 60% percoll (2 ml per spleen). This was overlaid with 2 ml of HBSS and centrifuged at 2000 rpm for 20 min. The interface was harvested and the cells were washed twice. They were then resuspended in RPMI (with 10% FCS) and transferred to large Petri dishes and incubated for 90 min at 37° C. The non-adherent cells were removed and the adherent cells were cultured overnight (approx 18 hr) with 1 ng/ml of GM-CSF and 1 micromolar SIINFEKL peptide (SEQ ID NO:1). The non-adherent cells were harvested the next day by gently pipetting and the cells were washed. B cell contaminants in this population are removed using goat anti mouse IgM and goat anti mouse IgG magnetic beads (Qiagen). The peptide loaded DC-rich cell preparation was then injected ip into mice (1×10⁶ cells per mouse). On an average 60-65% of the cells stained positive for markers characteristic of DC; CD11c, MHC Class II, CD80 and CD86.

Adoptive transfer and in vivo activation: Single cell suspensions were made from the spleen and peripheral lymph nodes of OT1 transgenic mice by mechanical homogenisation. RBCs were removed by density gradient centrifugation (Lympholyte-M, Cedarlane laboratories Ltd, Hornby, Ontario Canada). CD8+ T cells were purified by depletion of the MHC class II positive dendritic cells, B cells, macrophages using an Ab cocktail (clone 212.A1) specific for MHC class II molecules, clone 2.4-G2 specific for FcRs, Clone TIB 146 specific for B220, Clone GK1.5 specific for CD4, and Clone HB191 specific for NK1.1 marker). Magnetic beads coated with the secondary Ab were used to remove the cells coated with the primary Abs. Five million OT1 T cells (>90% pure CD8) were injected intravenously into recipient mice. The mice were activated with peptide loaded APCs injected intraperitoneally 24 hrs after injection of the OT1 cells.

Intracellular staining: Lymphocytes were isolated from the spleen, lymph nodes and livers of the different groups of mice, and about 2×10⁶ lymphocytes were either unstimulated or restimulated with 1 micromolar SIINFEKL peptide (SEQ ID NO:1) in complete medium with 50 unit/ml of recombinant mouse IL-2 (Endogen, Rockford, Ill.) and 1 microliter/ml of Golgi Plug™ (BD Biosciences Pharmingen, San Diego, Calif.)) in 96 well plates. After 6 hours of culture the cells were washed and stained for surface markers. They were then fixed and permeabilized using the BD Cytofix/Cytoperm™ kit and intracellular staining was performed according to the manufacturers instructions.

In vivo cytotoxicity assay: Splenocytes were isolated from WT C57BL10/SnJ mice and subjected to a Lympholyte gradient to eliminated the RBCs. One half of the cells were labeled with 2 microM CFSE (CFSE^{high}) and the other half was labeled with 0.2 microM CFSE (CFSE^{low}) for 10 minutes followed by two washes with PBS. The CFSE^{high} cells were pulsed with 1 microM SIINFEKL peptide (SEQ ID NO:1) for 1 hour at 37° C. while the CFSE^{low} cells were left unpulsed. The cells were washed extensively, counted and equal numbers of the two different populations were mixed together and injected intravenously into mice. About 10×10⁶ cells of each of the target groups was injected per mouse. The mice were sacrificed 5 hours later and the various organs were harvested.

vSAG-7 mediated activation: Splenocytes were isolated from AKR/J strains of mice and subjected to a Lympholyte gradient. Ten×10⁶ AKR/J splenocytes were injected into either C3H/HeJ (TLR-4 mutant) mice or C3H/HeOuJ mice (WT) mice. The AKR/J splenocytes express the vSAG-7 protein and can activate the Vbeta6+ T cells in the host. Spleen, lymph nodes and liver lymphocytes were isolated from the C3H/HeJ or C3H/HeOuJ strains of mice at various time points after transfer of the AKR/J splenocytes.

Cell isolation, staining and flow cytometric analysis: Peripheral lymph nodes and spleens were isolated from the mice on days 3, 5 and 7 after injection of pulsed or unpulsed DCs. Single cell suspensions were obtained by mechanical homogenization using frosted glass slides. The livers were perfused before they were harvested and intrahepatic lymphocytes (IHLs) were isolated using a standard protocol. Briefly, the livers were homogenized and treated with collagenase (0.05%) and DNAase (0.002%) for 45 minutes at 37° C. The hepatocytes were removed by low speed centrifugation (30 g for 5 min) and the remaining cell suspension was washed and subjected to an Optiprep gradient (Accurate Chemicals, Long Island, N.Y.). The Optiprep was used at a final concentration of 22% mixed with the cell suspension. This was overlaid with 2 ml of serum-free medium and centrifuged at 1500×g for 20 minutes at 4° C. The cells in the interface were isolated, washed and counted as IHLs.

Statistical analysis: Statistical significance of the differences observed between groups of mice was tested using the Student's t test. P values less than 0.05 were considered significant.

### Materials and Methods for Examples 6-10

Mice: C57BL/10ScN (TLR-4 deficient), C57BL/10SnJ (WT) mice, and OT1 transgenic mice were obtained and cared for in the manner described above. All mice were used between 6-8 weeks of age.

Adoptive transfer of OT1 cells: Single cell CD8+ T cell suspensions were obtained from OT1 transgenic mice and purified as described above. Five×10⁶ OT1 T cells (>90% pure CD8+) were injected intravenously into recipient mice.

Primary activation: The mice were activated with peptide loaded APCs injected intraperitoneally 24 hrs after injection of the OT1 cells. Dendritic cells were enriched from the spleen using the technique established by Livingstone, (“Isolation of CD4+ and CD8+ T Cell Clones from Mice Immunized with Synthetic Peptides on Splenic Dendritic Cells,” Methods 9:422-9 (1996), which is hereby incorporated by reference in its entirety) and used as APCs. The number of APCs injected was normalized for the percentage of CD11c+ cells, such that each mouse received about 0.5×10⁶ CD11C+ cells.

Secondary activation: Six weeks after primary immunization with peptide pulsed APCs, mice were challenged with SIINFEKL peptide (SEQ ID NO:1) in saline injected intraperitoneally. Three doses of SIINFEKL peptide (25 nmol each) were given every 24 hours. The mice were sacrificed and various organs were harvested 24 hours after the last dose of peptide (i.e., day 3 after the first peptide dose).

Isolation of liver lymphocytes: The livers were perfused before they were harvested and IHLs were isolated using the protocol standardized before. Briefly, the livers were homogenized and treated with collagenase (0.05% w/v) and DNAase (0.002% w/v) for 45 minutes at 37° C. The hepatocytes were removed by low speed centrifugation (30×g for 5 min) and the remaining cell suspension was washed and they were subjected to an Optiprep gradient (Accurate Chemicals, Long Island, N.Y.). The Optiprep was used at a final concentration of 22% (w/v Iadixanol), mixed with the cell suspension.

Isolation and transfer of OT1 memory cells: Six weeks after transfer of OT1 cells and primary stimulation with peptide pulsed DCs, CD8+ T cells were isolated from mice by negative depletion as described for the primary T cells. The memory cells were pooled from multiple mice in each group (WT or TLR-4 deficient) and the percentage of CD45.1+ cells was assessed in each case. The total cell number was adjusted such that all the mice received about 0.5×10⁶ OT1 memory cells (CD45.1+CD8+).

CFSE labeling: Cells were washed and resuspended in PBS (1×10⁷ cells/ml). CFSE was added at a final concentration of 5 μM. The cells were incubated for 10 min at 37° C., followed by two washes with HBSS.

Intracellular staining: Lymphocytes were isolated from the spleen, lymph nodes and livers of immune mice at the indicated time points, and about 2×10⁶ cells were cultured in complete medium containing 50 U/ml of rIL-2 (Endogen) and 1 microM Golgi Plug, in the presence or absence of antigen (1 microM SIINFEKL, SEQ ID NO:1). After 6 hours of culture the cells were washed and stained for surface markers. The cells were then fixed and permeabilized using the Cytofix/Cytoperm kit (BD Pharmingen) and intracellular staining was performed according to manufacturer's instructions.

Orthotopic liver transplantation: Orthotopic mouse liver transplantation was performed using the technique of (Steger et al., “Impact of Hepatic Rearterialization on Reperfusion Injury and Outcome After Mouse Liver Transplantation.” Transplantation. 76:327-332 (2003), which is hereby incorporated by reference in its entirety). The donor liver was exposed by a midline laparotomy and upper abdominal transverse incision. For continuous bile flow the gallbladder of the donor was removed after ligation at the root of the cystic duct. Following dissection of the surrounding hepatic ligaments, the right adrenal vein, pyloric vein, and proper hepatic artery were ligated and divided. A polyethylene stent tube (inner diameter 0.28 mm, outer diameter 0.61 mm; SIMS Portex, Kent, UK) was inserted into the lumen of the common bile duct and secured with 8-0 silk (Pearsalls, Taunton, UK). The infrahepatic inferior vena cava (IVC) and portal vein were clamped and the organ was perfused with 5 ml of 4° C. normal saline through the portal vein. The liver was removed to a 4° C. saline bath, a 20-gauge polyurethane cuff was placed at the portal vein stump, and the organ was retained at 4° C. until transplantation. Orthotopic liver transplantation was performed under isoflurane anesthesia. After clamping of the infra- and suprahepatic IVC and the portal vein, the recipient's liver was completely removed and the donor organ was placed orthotopically into the abdominal cavity. The supra- and the infrahepatic IVC were anastomosed with continuous running sutures using 10-0 nylon (Ethicon, Sommerville, N.J.), the portal vein was reconnected by cuff anastomosis. Reconstruction of the bile flow was achieved by inserting the graft's stent tube into the recipient's bile duct, and securing it with three single 10-0 nylon sutures.

Flow cytometric analysis: The stained cells were analyzed using a FACSCalibur (BD Biosciences, San Jose Calif.) and the data were analyzed using CellQuest software (BD Biosciences).

Statistical analysis: Statistical significance of differences between groups of mice was tested using either the student's t test (unpaired, two tailed) or using a 2×3 factorial or 2×4 factorial ANOVA for independent variables (VassarStats: available from the Vassar University Internet site). In all the cases, p<0.05 was considered significant.

### Example 1

### Liver is Defective in the Acute Trapping of Activated CD8+ T cells in the Absence of TLR-4

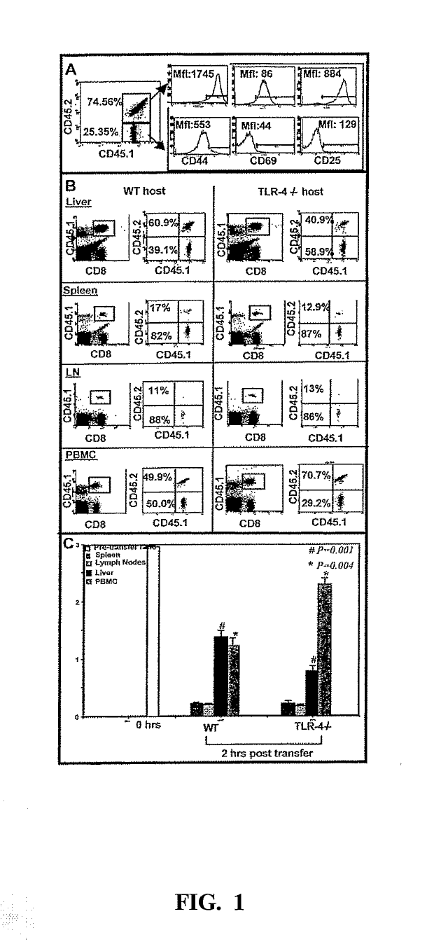

To test the role of TLR-4 in the accumulation of activated CD8+ T cells in the liver, a simple competitive trapping assay was developed to minimize any confounding effects of TLR-4 deficiency on CD8+ T cell activation or survival. In this assay, a mixture of activated and resting OT1 TCR transgenic CD8+ T cells was injected intravenously into either WT (C57 BL10/SnJ) or TLR-4 deficient mice (C57BL10Scn), and located by FACS 2 hours later. The activated and resting cells were identified based on their expression of distinct allotypes of CD45. FIG. 1A shows the expression of activation markers on the two cell populations. The activated cells (CD45.1+45.2+) seen in the upper right quadrant, expressed higher levels of CD44, CD69 and CD25 compared to the resting cells (CD45.1+, 45.2−) seen in the lower right quadrant.

At 2 hours after T cell injection into normal mice (marked WT host), resting CD8+ T cells localized preferentially to the spleen and lymph nodes, while activated CD8+ T cells were over represented in the liver (FIG. 1B, left panel). Injection of the same cell mixture into TLR-4 deficient mice (marked TLR-4−/− host) resulted in a different distribution of the cells (FIG. 1B, right panel). In the TLR-4 deficient mice, the preferential accumulation of the activated CD8+ T cells in the liver was lost, and conversely, such cells increased in the blood. FIG. 1C shows the ratio of activated to naïve CD8+ T cells in the spleen, lymph nodes, peripheral blood and liver of WT and TLR-4−/− mice (n=5 in each group). The ratio of activated to naïve cells in the livers of TLR-4−/− mice was significantly lower compared to WT mice. This decrease in the liver was compensated by a significant increase in this ratio in the peripheral blood of the TLR-4−/− mice. Therefore, TLR-4 promotes the removal of activated CD8+ T cells from the circulation, and their preferential localization in the liver.

### Example 2

### In Vivo Activation of CD8+ T Cells in Wildtype and TLR-4 Deficient Mice

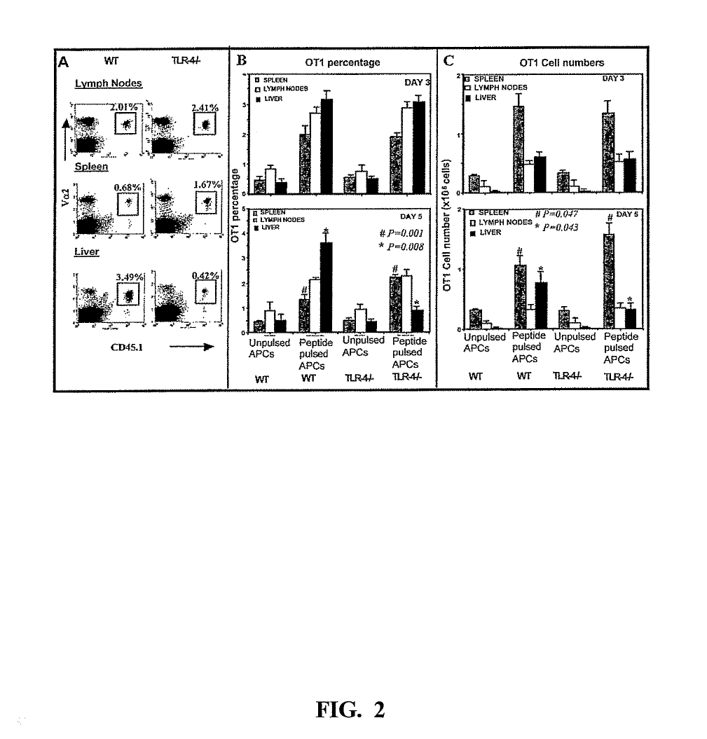

Testing the role of TLR-4 in the intrahepatic accumulation of CD8+ T cells activated in situ presents the problem that the TLR-4 deficient mice could be compromised in their ability to mount a normal immune response. Therefore, a model was developed in which normal OT1 TCR transgenic CD8+ T cells were transferred into either WT or TLR-4 deficient mice, and then primed in vivo using adoptively transferred spleen-derived dendritic cells from WT mice that had been pulsed in vitro with the specific antigenic peptide (SIINFEKL, SEQ ID NO:1). This model depends on direct priming, and the endogenous TLR-4 deficient APC are not involved (Wang et al., “Cutting Edge: CD4+ T Cell Help Can Be Essential For Primary CD8+ T Cell Responses in vivo,” J. Immunol. 171:6339-43 (2003), which is hereby incorporated by reference in its entirety). Using this model, equivalent clonal expansion of OT1 T cells was observed in the spleen and lymph nodes, but reduced accumulation of OT1 T cells was observed in the liver on day 5 of the response (FIGS. 2A-C). FIG. 2A shows individual examples of the frequencies of activated OT1 CD8+ T cells in different organs at day 5; the frequencies were similar in the lymph nodes of WT and TLR-4 deficient mice, but there were eightfold fewer OT1 T cells in the livers of the TLR-4 deficient mouse. The analysis of groups of mice (n=6) at day 3 showed that there were no significant differences in the percentages (FIG. 2B, top panel) and numbers (FIG. 2C, top panel) of OT1 cells in the spleen, lymph nodes, or livers of the WT and TLR-4 deficient mice at day 3, suggesting that the priming and clonal expansion of the OT1 cells was comparable between the two groups of mice. However, on day 5, there was a significant reduction in the percentage (FIG. 2B, lower panel) and numbers of OT1 cells (FIG. 2C, lower panel) in the livers of the TLR-4 deficient mice. The reduced accumulation of the activated OT1 cells in the livers of the TLR-4 deficient mice was accompanied by an increase in their percentages in the spleen (also seen in the representative example in FIG. 2A), suggesting that the cells that were not trapped in the liver were migrating to the spleen. Thus, the intrahepatic accumulation of activated CD8+ T cells during an in situ immune response is promoted by TLR-4.

### Example 3

### Activation of the OT1 Cells is Similar in Wildtype and TLR-4 Deficient Mice

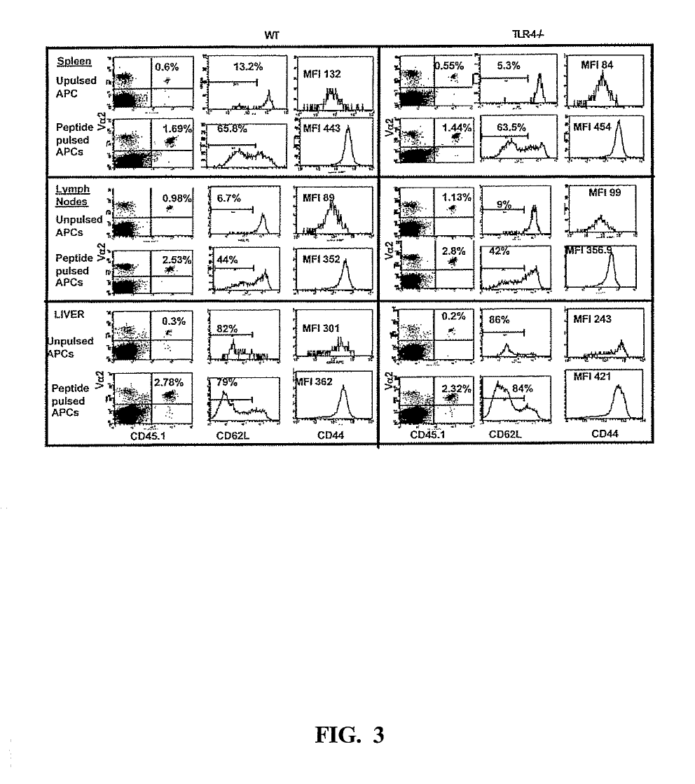

To address the issue of whether the reduction in the OT1 cell numbers seen in the TLR-4 deficient livers was a result of differential activation, the responses in the two groups of mice was examined more closely. FIG. 3 shows that at three days after antigen exposure, the OT1 T cells were activated normally in TLR-4 deficient mice. Thus, the cells showed equivalent clonal expansion (for example, in the spleen from 0.60% to 1.69% of all lymphocytes in B6 mice, and from 0.55% to 1.44% in TLR-4 deficient mice), and this was also true in lymph nodes and the liver. The down-regulation of CD62L and up-regulation of CD44 also occurred identically in the B6 and the TLR-4 deficient hosts (FIG. 3). This was not surprising, since the T cells residing in both the WT and TLR-4−/− mice were activated with dendritic cells from WT mice. Such equivalent activation confirm the accuracy of the conclusions drawn from the observed differences in the TLR-4−/− livers.

### Example 4

### Activated OT1 Cells in Wildtype and TLR-4 Deficient Mice can Produce IFN-Gamma and Kill Antigen-Loaded Targets In Vivo

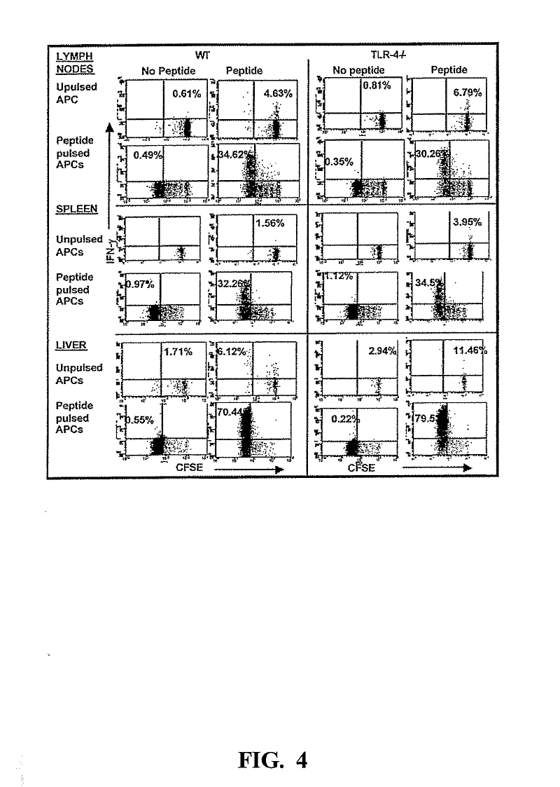

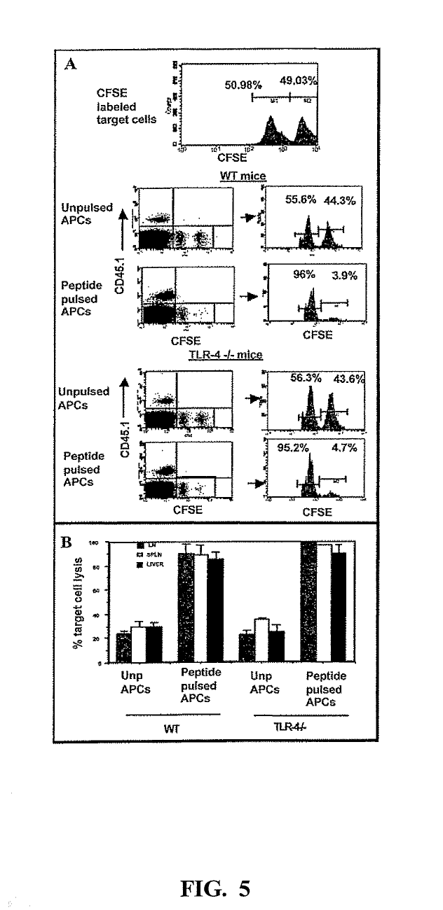

To validate the conclusion that OT1 cells were activated normally by WT DC in TLR-4 deficient hosts, an examination was performed to assess the ability of the OT1 cells activated in the normal and TLR-4 deficient mice to synthesize the effector cytokine IFN-gamma and to kill target cells in vivo. In the WT mice, OT1 T cells that were sham primed with PBS-pulsed APCs did not divide, and only a few cells (4.63% in the lymph nodes and 1.56% in the spleen) were competent to make IFN-gamma on restimulation in culture with the antigenic peptide (FIG. 4). The OT1 cells in the WT mice that were primed with peptide pulsed APCs had gone through at least six divisions by day 3, and they were capable of synthesizing IFN-gamma upon restimulation with SIINFEKL peptide (SEQ ID NO:1). The IFN-gamma production was antigen specific since it was seen only when the cells were restimulated with the antigenic peptide in the 6 hour in vitro assay. A comparison of the dilution of CFSE in the OT1 population from the WT and TLR-4 deficient mice revealed no significant differences in the number of cell divisions that occurred in the two different recipients. There was also no significant difference between the frequency of IFN-gamma producing OT1 cells, which were activated either in the WT or TLR-4−/− mice. The data shown in FIG. 4 are representative of 6 mice in each group. The hallmark of a fully functional effector CD8+ T cell is its ability to kill target cells expressing specific antigens and, hence, the cytotoxic capability of the OT1 T cells that were activated in the TLR-4 deficient mice was tested using an in vivo cytotoxicity assay. Specific targets (loaded with SIINFEKL peptide, SEQ ID NO:1) and non-specific target cells (not loaded with peptide) were labeled with two different concentrations of CFSE, so that they could be tracked in the various organs 5 hours later. FIG. 5A shows that, in normal mice, which received OT1 cells but were sham primed with PBS pulsed APCs, the ratio of the specific (CFSE^{high}) to non-specific (CFSE^{low}) targets in the lymph nodes was comparable to the ratio of the same before injection. On the other hand, the mice in which the OT1 cells were primed with peptide pulsed APCs showed a reduction in the percentage of the CFSE^{high} targets. This shows that the activated OT1 cells were cytotoxic and specifically killed the peptide loaded targets. In the TLR-4−/− mice, which were immunized with peptide pulsed APCs, there was a similar specific loss in the peptide loaded target cell population (FIG. 5A, bottom panel). FIG. 5B shows the percentage of specific target cell lysis in the spleen, lymph nodes, and livers of WT and TLR-4 deficient mice (n=6 in each group). The extent of loss of the specific target cells was similar between the WT and TLR-4−/− mice in all the three organs tested, suggesting that there was no difference in the cytotoxic activity of the OT1 cells activated in the two different recipients.

The identical activation and function of the OT1 cells in the WT and TLR-4−/− mice suggested that the reduced accumulation of these cells in the livers of TLR-4 deficient mice later in the immune response was the result of a local effect of TLR-4 in the liver on trapping, rather than a systemic effect on priming.

The reduced percentage of OT1 cells seen in the TLR-4−/− liver at day 5 could be attributed to an increased death rate of these cells. To test this possibility, the percentage of dying cells was estimated by measuring caspase-3 activity. Caspase-3 is downstream of both the active (death receptor mediated) and passive (mitochondrial) death pathways and, hence, is an indication of the total cell death irrespective of the mechanism. Experiments showed that there was no difference in the percentage of caspase-3 positive OT1 cells at day 3 or day 5 between the WT and TLR-4−/− mice. This confirmed that the decrease in the percentage and numbers OT1 cells seen in the TLR-4 deficient mice was not a result of a higher rate of apoptosis of these cells in the TLR-4 deficient mice.

### Example 5

### TLR-4 Mutant Mice Show Similar Lack of Trapping of vSAG7 Activated Cells

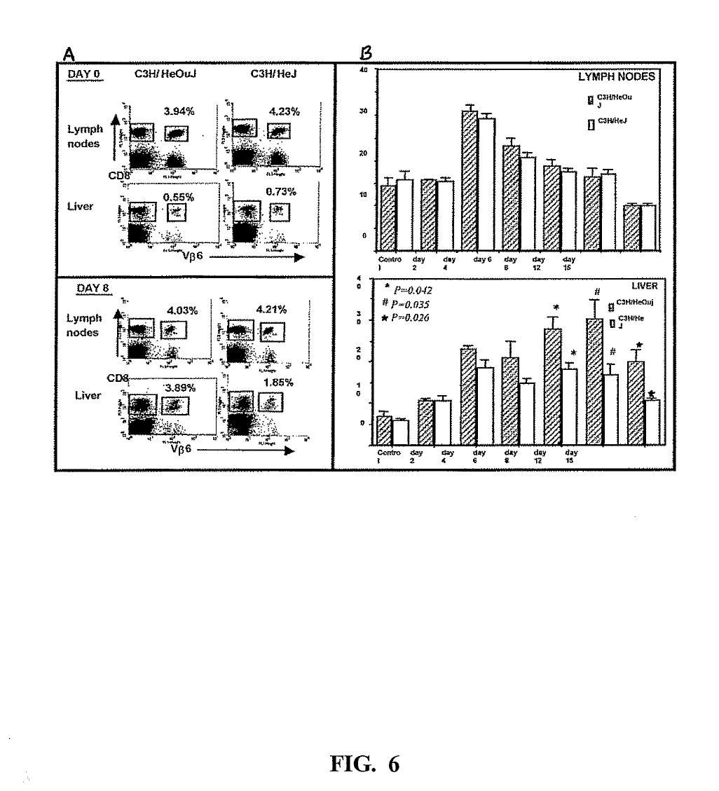

The data from the preceding examples suggest that the liver loses its ability to trap activated CD8+ T cells efficiently. To test if this was dependent on the capacity of TLR-4 to engage downstream signaling pathways, the TLR-4 mutant strain, C3H/HeJ was used. Polyclonal activation of Vbeta6+T cells was induced in two strains of C3H mice: the C3H/HeJ (TLR-4 mutant) strain and C3H/HeOuJ (normal TLR-4) strain. In both cases, Vbeta6 T cells were activated with an injection of AKR/J spleen cells, which express the endogenous retrovirus Mtv-7, encoding the superantigen vSAG-7. This procedure causes activation, followed by deletion of both CD4+ and CD8+ T cells expressing Vbeta6 as a part of the TCR (Huang et al., “Superantigen-driven Peripheral Deletion of T Cells. Apoptosis Occurs in Cells That Have Lost the alpha/beta T Cell Receptor,” J. Immunol. 151:1844 (1993), which is hereby incorporated by reference in its entirety). FIG. 6A shows the representative frequencies of Vbeta6 CD8+ T cells in the lymph nodes and livers of WT and TLR-4 mutant strains mice on day 0 (pre-immunization) and day 8-post immunization. On day 0 the percentage of the Vbeta6 CD8+ T cells was comparable in both the lymph nodes and livers of the two different strains of mice. However on day 8, fewer activated Vbeta6 CD8+ T cells were seen in the liver of the TLR-4 mutant mice. FIG. 6B shows the average percentage of Vbeta6 CD8+ T cells (as a percentage of the total CD8+ T cell percent) over time in the lymph nodes and livers of the TLR-4 mutant and WT mice (n=6 at each time point for each of the groups). Both C3H/HeJ and C3H/HeOuJ strains of mice showed a comparable clonal expansion in their Vbeta6 CD8+ T cell population, followed by deletion in the lymph nodes (FIG. 6B) and spleen over a period of 15 days (top panel). This deletion from the periphery was accompanied by the accumulation of the cells in the liver. TLR-4 mutant mice showed lower accumulation of Vbeta6 CD8+ T cells in the liver compared to WT mice, which was more apparent and significant on days 8 and 12. The Vbeta6 CD4+ T cells also went through activation and deletion, but few of these cells accumulated in the liver and no significant difference was seen between the two strains of mice.

These data argue that TLR-4 does not affect vSAG induced T cell activation in the lymphoid organs, but is involved in promoting the accumulation of activated CD8+ T cells in the liver. These data support the short term trapping experiments and the in vivo priming experiments in the TLR-4 deficient mice. In all three experimental models, TLR-4 promotes the trapping of activated CD8+ T cells in liver.

### Discussion of Examples 1-5

The extensive literature on TLRs emphasizes their role in augmenting and initiating innate immune responses. Thus, TLRs are involved in the maturation of specialized antigen presenting cells such as dendritic cells, the induction of co-stimulatory molecules, production of cytokines and chemokines by the cells of the innate immune system, and in the resistance of DC to regulatory T cells (Iwasaki et al., “Toll-like Receptor Control of the Adaptive Immune Responses,” Nat. Immunol. 5:987 (2004); Takeda et al., “Toll-like Receptors,” Annu. Rev. Immunol. 21:335 (2003), each of which is hereby incorporated by reference in its entirety). However, in recent years several other aspects of TLR biology have emerged. In the liver, antigen presentation is strongly influenced by LPS but in an unexpected way; endotoxin down-regulates T cell activation by LSECs and the CD4+ and CD8+ T cells that are activated by LSECs show a tolerant phenotype (Knolle et al., “Liver Sinusoidal Endothelial Cells Can Prime Naive CD4+ T Cells in the Absence of IL-12 and Induce IL-4 Production in Primed CD4+ T cells: Implications for Tolerance Induction in the Liver,” Gastroenterology 116:1428 (1999); Limmer et al., “Efficient Presentation of Exogenous Antigen by Liver Endothelial Cells to CD8+ T Cells Results in Antigen-specific T-Cell Tolerance,” Nat. Med. 6:1348 (2000), each of which is hereby incorporated by reference in its entirety). Thus, in this context, TLR engagement is immunosuppressive. Similarly, LPS acting on Kupffer cells and LSECs leads to the secretion of the immunosuppressive mediators such as IL-10 and TGF-beta (Knolle et al., “Control of Immune Responses by Scavenger Liver Endothelial cells,” Swiss Med Wkly. 133:501 (2003), which is hereby incorporated by reference in its entirety).

More recently, the recognition of commensal-derived products by TLRs has been shown to play an important role in normal intestinal epithelial homeostasis (Rakoff-Nahoum et al., “Recognition of Commensal Microflora by Toll-like Receptors Is Required for Intestinal Homeostasis,” Cell 118:229 (2004), which is hereby incorporated by reference in its entirety). The data indicate a different function for TLR-4 under non-inflammatory conditions; TLR-4 ligands, possibly from the normal enteric flora, have a direct effect on the ability of the liver to trap activated CD8+ T cells.

The central issue in experiments designed to test ideas concerning the influence of the TLR-4 on the distribution of circulating CD8+ T cells is the concern that TLR-4 deficient mice might have defects in priming outside the liver, which might have secondary consequences for intrahepatic trapping of cells. To address this concern, a very simple direct short term in vivo localization assay was adopted, where a mixture of activated and naïve CD8+ T cells was intravenously transferred into either TLR-4−/− or normal mice and located by FACS at 2 hours. This leads to differential partitioning in the different tissues in a normal mouse. Naïve CD8+ T cells were preferentially localized in the spleen and lymph nodes while the activated cells were predominantly in the liver. In the absence of TLR-4 fewer activated CD8+ T cells were extracted from the peripheral blood into the liver, indicating that TLR-4 signaling promotes the localization of the circulating activated CD8+ T cells to the liver. The diagnostic feature of the liver specific effect in this assay is the change in the abundance of activated versus resting CD8+ T cells in the liver and a reciprocal effect in the blood. It is quite possible that in the absence of TLR-4, the adhesion mechanisms in the periphery are also defective. However, the fact that there were no differences in the small fraction of activated CD8+ T cells that had migrated into the spleen and lymph nodes of the WT and the TLR-4−/− mice suggests otherwise. In this experimental model, recipient mice only interact with the input cells for 2 hours of the assay, which emphasizes effects on T cell localization over considerations such as priming and survival.

To interpret the consequences of the lack of trapping of activated CD8+ T cells in the liver, it was imperative to test this effect in an in situ immune response. However, it was also important to control for the known and unknown defects in priming in the TLR-4 deficient mice. To achieve this, OT1 cells were adoptively transferred into either WT or TLR-4 deficient mice and primed using wild type peptide pulsed APCs. The clonal expansion and proliferation of the OT1 cells that were activated either in the WT or TLR-4 deficient mice were comparable at day 3. However at five days fewer of the activated CD8+ T cells were retained in the livers of the TLR-4 deficient. The hypothesis that there was greater apoptosis of the OT1 cells in the absence of TLR-4 in the liver was tested. The lack of any significant differences in the percentage of Caspase-3 positive OT1 cells in the liver, spleen, or lymph nodes of the WT and TLR-4 deficient mice indicated that the TLR-4 effect could not be attributed to differential apoptosis of the OT1 cells. The functional competence of the OT1 cells that were activated in either the WT or TLR-4 deficient mice was also examined and found that they were identical in terms of their ability to produce IFN-gamma and in their cytotoxicity. All of this suggested that the lower numbers of OT1 cells seen in the liver in the absence of TLR-4 is, in fact, due to a difference in the trapping and retention in the liver, rather than an effect of differential priming or survival of these cells elsewhere. The compensatory increase in the percentage of the OT1 cells in the spleens of the TLR-4−/− mice is further evidence for this.

In adoptive transfer experiments, OT1 transgenic T cells have been transferred, which are on a C57BL/6 background, into C57BL/10 congenic recipients. The substrains 6 and 10 of C57BL mice (C57BL/6 and C57BL/10) differ at a few minor histocompatibility antigen loci. However, no difference in the survival was noticed (up to 10 weeks) or activation status of OT1 transgenic cells in the absence of any stimulation, when transferred into either C57B1/6J or C57B1/10SnJ mice. Hence, the conclusion is that the use of C57BL16 T cells in C57BL/10 congenic hosts did not compromise the experiments.

To test whether the observed effect was due to signaling downstream of TLR-4 in a normal liver, the TLR-4 mutant mouse strain (C3H/HeJ) was used, which can bind LPS but cannot signal through it. Using a different model of activation (superantigen encoded by an endogenous retrovirus), the TLR-4 mutant mice still accumulated fewer activated cells compared to the WT mice. Both in the vSAG-7 mediated activation of the Vbeta6 CD8+ T cells and in the activation of OT1 cells by SIINFEKL (SEQ ID NO:1) pulsed APCs, the difference in the accumulation of the activated CD8+ T cells in the TLR-4 mutant or deficient livers was seen at the later phases of the response. In the TLR-4 mutant mice, up to day 8 the accumulation of Vbeta6 T cells in the liver was comparable to the control mice. It is when the response began to fade in the periphery that the difference in accumulation in the liver was more apparent.

The current model to explain these observations is: (a) commensal derived products from the gut engage TLR-4 in the liver; (b) TLR-4 signaling promotes the expression of adhesion molecules; (c) activated CD8+ T cells are retained in the hepatic sinusoids due to these adhesion mechanisms; and (d) such sequestration removes them from the circulating pool.

### Example 6

### TLR-4 Deficient Mice Show a Higher Frequency of the CD8+ T Cell Memory Precursors Compared to Wildtype Mice

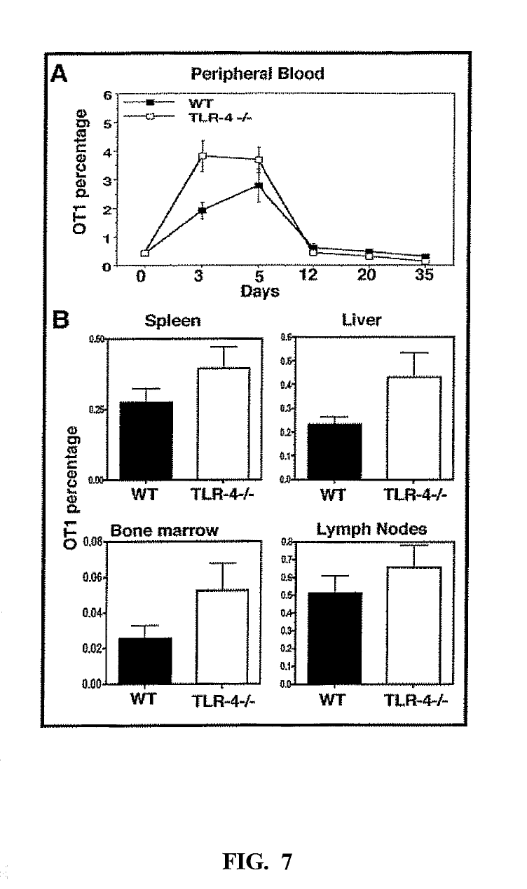

Data presented in Examples 1-5 show that TLR-4 regulates the trafficking of activated CD8+ T cells to the liver (see also John et al., “TLR-4 Regulates CD8+ T Cell Trapping in the Liver,” J Immunol 175:1643-50 (2005), which is hereby incorporated by reference in its entirety). It was expected, therefore, that reduced trapping of activated CD8+ T cells in the liver of TLR-4 deficient mice would make more cells available to enter the peripheral pool of primed CD8+ T cells. To test this, normal versus TLR-4 deficient mice were given an adoptive transfer of OT-1 T cells and immunized with antigen-loaded dendritic cells (DC). To compensate for any deficiencies in priming due to the absence of TLR-4, APCs from WT mice loaded with SIINFEKL peptide (SEQ ID NO:1) were used to prime the OT1 cells in both the WT and the TLR-4 deficient mice. The circulating primed OT1 cell population was monitored in the peripheral blood of WT and TLR-4 deficient mice over a period of six weeks (FIG. 7A). Using such a priming technique, it was shown in the preceding examples that the activation of cells was identical between the WT and the TLR-4 deficient mice, and that TLR-4 deficient mice showed reduced percentage and numbers of OT1 cells in their livers at day 5 (see also John et al., “TLR-4 Regulates CD8+ T Cell Trapping in the Liver,” J Immunol 175:1643-50 (2005), which is hereby incorporated by reference in its entirety). On days 3 and 5 after the immunization, there were a higher percentage of OT1 cells in the peripheral blood of TLR-4 deficient mice compared to the WT mice (FIG. 7A). This is consistent with the lack of trapping of these cells in the liver, as described above. However, at later time points (days 12, 20, 35) the percentage of OT1 cells in the blood was not significantly different between the WT and TLR-4 deficient mice, suggesting that the cells that were not trapped in the liver were migrating to other peripheral sites. To test this, a number of peripheral lymphoid and non-lymphoid compartments were examined six weeks after immunization for the presence of the primed OT1 CD8+ T cells. The OT1 cells (CD45.1+CD8+) were more abundant in the spleen, liver, bone marrow, and lymph nodes of the TLR-4 deficient mice compared to the WT mice (FIG. 7B). The difference in the percentage of OT1 cells at six weeks was significantly different (p=0.025) between the WT and TLR-4 deficient when tested using a 2×4 factorial ANOVA, for all the four tissues (liver, spleen, lymph nodes, and bone marrow) additively. This difference was also apparent in the absolute number of OT1 cells. The conclusion is that there is an indirect correlation between the early trapping of activated CD8+ T cells in the liver and the size of the primed OT1 T cell population seen in the periphery at the later phases.

### Example 7

### CD8+ Memory Precursors Generated in Wildtype and TLR-4 Deficient Mice are Functionally and Phenotypically Identical

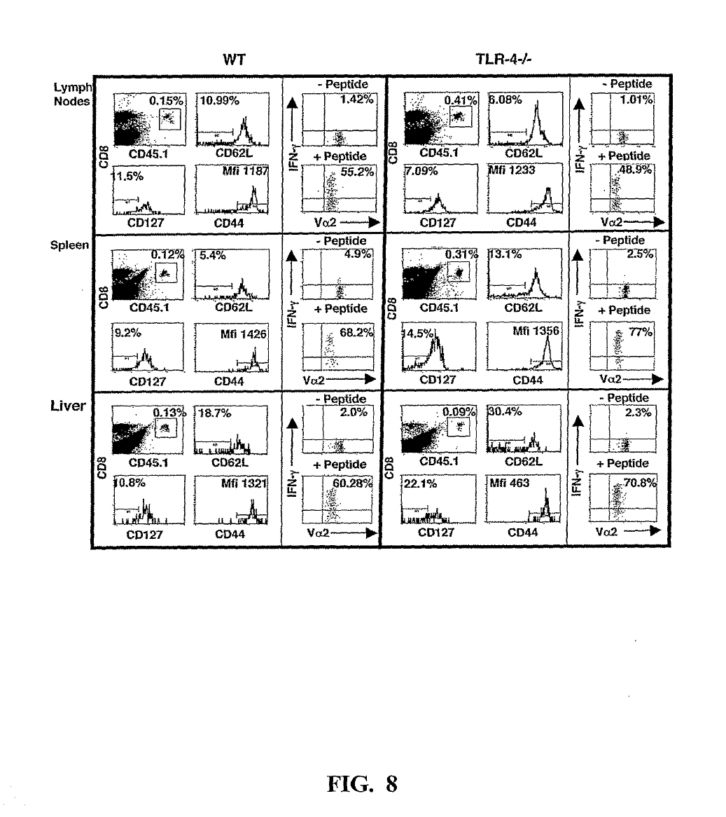

To address the issue of whether the primed OT1 cells found in the TLR-4 deficient mice 6 weeks after primary immunization were true memory precursors, and to test whether they were qualitatively different from the memory cells generated in the WT mice, the phenotype and function of these cells were examined. The OT1 cells isolated from the spleen, lymph nodes, and livers of the WT mice (FIG. 8) expressed CD62L, CD44, and CD127 at high levels on their surface, and showed low forward and side scatter, all of which together confirmed that they were an ‘antigen experienced’ resting population of cells. There was, however, no difference between the OT1 cells isolated from the WT or TLR-4 deficient mice with respect to expression of the surface markers tested.

To test whether these cells were functionally competent memory T cells, they were assayed for their ability to produce IFN-gamma. Isolated OT1 cells, re-stimulated in culture with the antigenic peptide, produced IFN-gamma and there was no significant difference in the ability of cells from WT versus TLR-4−/− hosts to synthesize this cytokine (FIG. 8). The OT1 cells isolated from WT and TLR-4 deficient mice were comparable to one another with respect to their ability to proliferate in vitro. No difference was observed between the WT and TLR-4 deficient mice in their ability to lyse target cells specifically, which was assayed by an in vivo cytotoxicity assay described in the preceding Materials and Methods section. This indicates that equally cytotoxic OT1 memory cells were generated in the WT and TLR-4 deficient mice.

Based on the expression of surface markers, the ability to proliferate, synthesize IFN-gamma, and lyse specific target cells, the conclusion is that the memory precursors generated in the WT and TLR-4 deficient mice are qualitatively similar.

### Example 8

### TLR-4 Deficient Mice Make Larger Recall Responses than Wildtype Mice

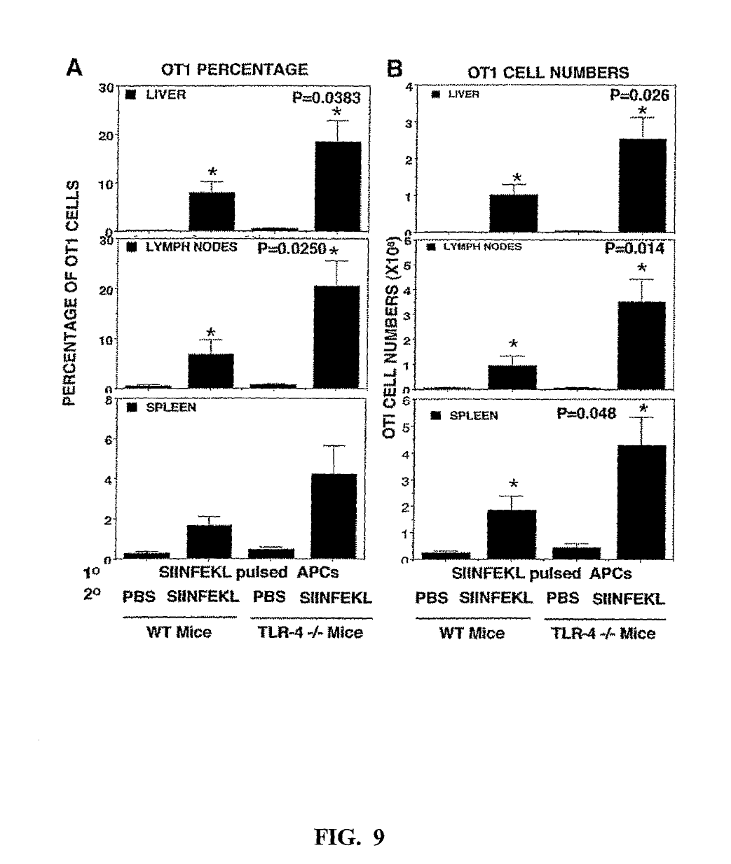

To address the issue of whether the higher frequency of memory precursors seen in the TLR-4 deficient mice results in increased recall responses, OT1 cells that were primed in WT or TLR-4 deficient mice with antigenic peptide in saline were challenged six weeks after primary immunization with peptide pulsed APCs. The clonal expansion of OT-1 T cells, 3 days after re-stimulation with antigenic peptide, was used as a measure of memory CD8+ T cell function. In wild-type mice, trace numbers of OT-1 T cells were detected in mice injected with saline (FIG. 9A, PBS). When such mice were challenged with peptide there was a detectable secondary response, with OT-1 T cells expanding to around 1.6% of the spleen, and 7-8% of the lymph nodes and the liver (FIG. 9A, SIINFEKL). In the TLR-4 deficient mice, there were more OT-1 T cells in the PBS-challenged controls, particularly in the lymph nodes and spleen (FIG. 9A, PBS). When such mice were challenged with peptide, there was increased abundance of OT-1 T cells, with approximately 4% in the spleen, and about 18-20% in the lymph nodes and liver; these differences were statistically significant (P<0.05 in all cases). The absolute OT1 cell numbers (FIG. 9B) revealed the same difference (P<0.05 in all cases). Thus, there was a 2.5 fold increase in both the percentage (FIG. 9A) and the numbers (FIG. 9B) of OT1 T cells in the TLR-4 deficient hosts. The conclusion is that in TLR-4 deficient hosts there is increased survival of activated CD8+ T cells during the early phase of the response, resulting in more memory T cells and, hence, a larger secondary response.

### Example 9

### Higher Precursor Numbers and Greater Secondary Expansion are Responsible for Larger Secondary Responses in the TLR-4 Deficient Mice

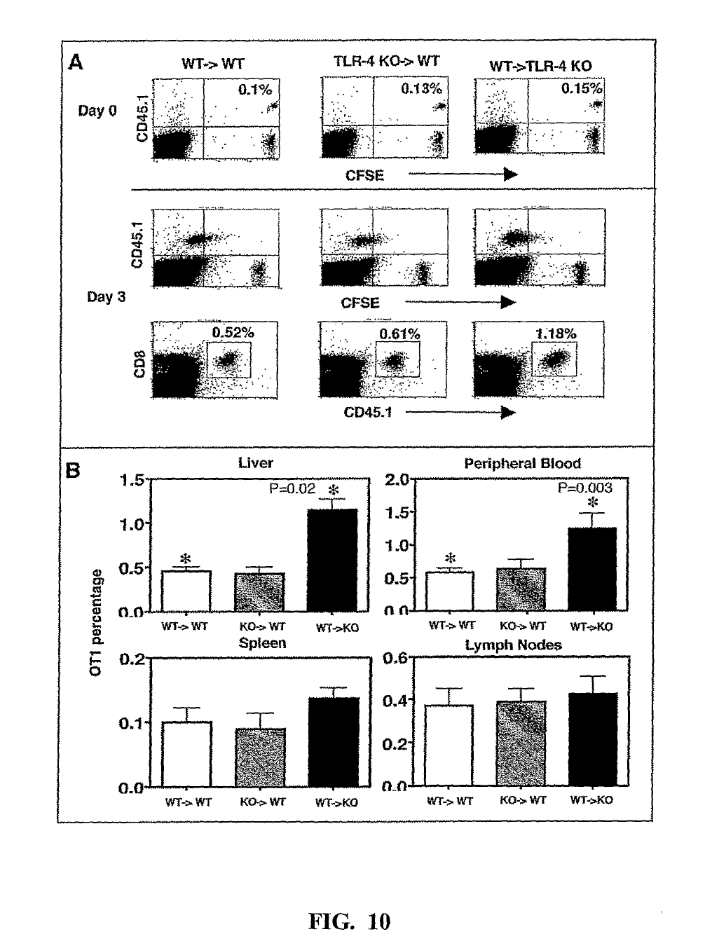

The data shows that higher secondary responses in the TLR-4 deficient mice were associated with the higher percentage of memory cells in these mice. Multiple tests show that the quality of the memory cells generated in the TLR-4 deficient mice was not different either phenotypically or functionally from the memory cells generated in the WT mice. If the higher memory cell numbers were the sole reason for the increased recall responses seen in the TLR-4 deficient mice, then eliminating the difference in precursor numbers would take away the difference in the secondary response between the WT and TLR-4 deficient mice. To test this, CD8+ T cells were isolated from WT or TLR-4 deficient mice 6 weeks after primary immunization with peptide pulsed APCs, and injected equal numbers of CD45.1+CD8+ T cells into new recipients. The transferred cells were labeled with CFSE, and upon restimulation with antigenic peptide in vivo the OT1 cells (CD45.1+CFSE+) divided specifically, as seen by the dilution of CFSE, whereas the antigen non-specific CD8+ T (CFSE+CD45.1−) cells did not divide (FIG. 10A, Day 3). Whether they were derived from WT mice or from TLR-4 deficient mice, the OT1 memory cells divided to the same extent upon transfer into WT recipients (WT->WT or TLR-4->WT) (FIG. 10A). The total percentage of OT1 cells in the peripheral blood before (day 0) and 3 days after restimulating with antigenic peptide (day 3) was equivalent whether the WT mice received memory OT1 precursors generated in the WT or in the TLR-4 deficient mice. An average of the percentage of OT1 cells in the other lymphoid and non-lymphoid compartments such as spleen, lymph nodes, and liver (FIG. 10B) indicated that, on a per cell basis, there was no difference in the secondary responses obtained from OT1 memory precursors whether they were generated in the WT or the TLR-4 deficient mice.

In contrast to these results, when the OT1 memory precursors primed in the WT mice were re-transferred into a TLR-4 deficient recipient (WT->TLR-4), and re-stimulated with antigenic peptide, a larger secondary response was generated (FIG. 10A) compared to the responses seen when the recipient was a WT mouse (WT->WT). This difference was most striking in the liver and the peripheral blood (FIG. 10B). These data suggest that the higher recall response seen in the TLR-4 deficient mice were not completely explained by the higher frequency of precursors. Instead, an additional conclusion is that the presence of TLR-4 has a negative effect on the process of secondary expansion in vivo. The difference in the precursor numbers between the intact WT and intact TLR-4 deficient mice was 1.5 fold, and the difference in the secondary responses between the intact WT and TLR-4 deficient mice was about 2.5 fold. When an equal number of memory precursors were re-transferred into WT or TLR-4 deficient mice, the difference in the expansion was about 2 fold. This suggests that the effect on the difference in precursor numbers and the effect on secondary expansion between the WT and the TLR-4 deficient mice were additive.

### Example 10

### Liver is the Site of the TLR-4 Dependent Effect on Secondary Responses

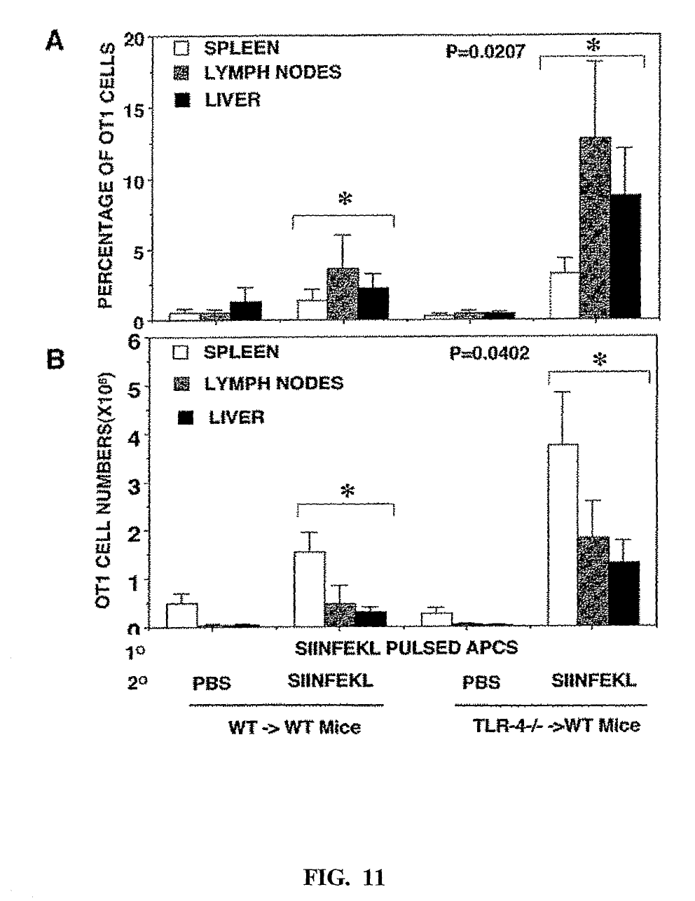

The data from the preceding examples show that in the absence of TLR-4, activated cells are trapped to a lesser extent in the liver and this, in turn, leads to a higher percentage of primed cells that are available to contribute to the memory pool. To test the importance of TLR-4 expressed in the liver on memory responses, the most direct approach was adopted: livers of WT or TLR-4-deficient mice were transplanted orthotopically into wild-type hosts. This involves the removal of the recipient liver, and the grafting of the donor organ with reconstruction of the vena cava, portal vein and bile duct. After 4 weeks, the operation was fully healed and the recipient mice were healthy. Such mice received an adoptive transfer of OT1 T cells and were primed with peptide-loaded DC. Six weeks later, the transplanted, primed mice were challenged with antigenic peptide in saline. In WT mice that received a normal B6 liver (WT->WT), a detectable CD8+ T cell secondary clonal expansion was elicited as seen in the increase in the percentage of OT1 cells in the spleen, lymph nodes and liver between the PBS challenged and SIINFEKL peptide challenged mice (FIG. 11A, left side). In contrast, in WT recipient carrying a TLR-4 deficient liver (TLR-4->WT), a larger expansion was observed in response to antigenic peptide (FIG. 11A, right side). There was a higher percentage (FIG. 11A) and absolute number (FIG. 11B) of OT 1 T cells in all the compartments (spleen, liver, lymph nodes) of the WT mice that received a TLR-4 deficient liver compared to the WT mice that received a WT liver. The conclusion is that the effect of TLR-4 on the formation of CD8+ T cell memory is mediated in the liver itself.

### Discussion of Examples 6-10

The liver is a unique tolerance-inducing organ but is also capable of sustaining effective immune responses to pathogens, which suggests that a complex interplay of various factors shifts the balance towards either intrahepatic tolerance or immunity (Bowen et al., “Intrahepatic Immunity: A Tale of Two Sites?,” Trends Immunol 26:512-7 (2005); Crispe, “Hepatic T Cells and Liver Tolerance,” Nat Rev Immunol 3:51-62 (2003), each of which is hereby incorporated by reference in its entirety). It has been shown that the liver also plays an important role in clearing activated CD8+ T cells at the end of a systemic CD8+ T cell response (Crispe et al., “The Liver as a Site of T-Cell Apoptosis: Graveyard, or Killing Field?,” Immunol Rev 174:47-62 (2000), which is hereby incorporated by reference in its entirety). Although this process occurs in diverse kinds of immune responses, the consequences of such trapping on the long-term immune response were not previously understood.

It has been shown from Examples 1-5 that TLR-4 signaling promotes the localization of circulating activated CD8+ T cells to the liver both in a short-term homing experiment and during an in situ immune response (see also John et al., “TLR-4 Regulates CD8+ T Cell Trapping in the Liver,” J Immunol 175:1643-50 (2005), which is hereby incorporated by reference in its entirety). The effect of TLR-4 in the liver was primarily on trapping and not on the apoptosis of intrahepatic CD8+ T cells. This is based on the fact that the frequency of apoptotic cells among the activated OT1 cells at the peak of liver accumulation was not different between the WT and TLR-4 deficient mice; however, since fewer cells were trapped in the livers of the TLR-4 deficient mice, there were fewer dying cells in the liver and this explains the greater percentage of cells seen in the peripheral circulation. Although the difference in each of the peripheral tissues tested was small, additively, there was a significantly higher percentage of total memory precursors in the TLR-4 deficient mice that can respond to a restimulation at 6 weeks compared to the WT mice. Current data clearly indicated that the lack of TLR-4 results in a greater magnitude of secondary responses and the role of the liver in this process was revealed through the transplantation experiments.

Models for CD8+ T cell memory generation evolve constantly. There is now a consensus that the path to differentiation of memory CD8+ T cells involves three distinct stages, which also function as crucial checkpoints. The first phase is the expansion phase, where the CD8+ T cells need to be optimally activated to generate a large pool of effectors cells. The second phase is the contraction phase when the majority of the effectors cells die, and the third phase is the memory phase when the memory cell number is stabilized in different compartments and they are homeostatically maintained thereafter (Kaech et al., “Effector and Memory T-Cell Differentiation: Implications for Vaccine Development,” Nat Rev Immunol 2:251-62 (2002), which is hereby incorporated by reference in its entirety). Recent studies have indicated differences in the rate of apoptosis of activated cells that migrate to the lymphoid versus non-lymphoid compartments (Wang et al., “Virus-Specific CD8 T Cells in Peripheral Tissues are More Resistant to Apoptosis than those in Lymphoid Organs,” Immunity 18:631-42 (2003), which is hereby incorporated by reference in its entirety), which makes the migratory patterns of the activated CD8+ T cells important during the contraction phase. Activated CD8+ T cells can be isolated from a variety of non lymphoid compartments; however, based on experiments involving adoptive transfer of memory cells from various non lymphoid tissues (Masopust et al., “Activated Primary and Memory CD8 T Cells Migrate to Nonlymphoid Tissues Regardless of site of Activation or Tissue of Origin,” J Immunol 172:4875-82 (2004), which is hereby incorporated by reference in its entirety) and the use of parabiotic mice (Klonowski et al., “Dynamics of Blood-Borne CD8 Memory T Cell Migration in vivo,” Immunity 20:551-62 (2004), which is hereby incorporated by reference in its entirety), it is clear that activated/memory cells are capable of recirculation. Data indicate that the activated CD8+ T cells that fail to be retained in the liver are capable of recirculating back into the peripheral pool.

Based on the data, a modification to current models of CD8+ T cell memory generation is proposed, in which the liver plays a key role during the contraction phase. Once activated, CD8+ T cells traffic through various compartments, and when they pass through the liver a large fraction of them are retained there. The liver preferentially sequesters activated CD8+ T cells, and not simply T cells already undergoing apoptosis (Mehal et al., “Selective Retention of Activated CD8+ T Cells by the Normal Liver,” J Immunol 163:3202-10 (1999), which is hereby incorporated by reference in its entirety), suggesting that the sequestration starts as soon as activated CD8+ T cells leave priming sites and begin to circulate in the blood. In the liver, a proportion of the trapped CD8+ T cells are subjected to apoptosis. This model predicts that, at each passage through the liver, activated CD8+ T cells that have not entered either lymphoid or non-lymphoid tissues will be depleted. Thus, the liver acts as a “sink” for activated T cells that do not rapidly localize to either sites of infection, or sites where they can mature into long-lived memory cells. This interpretation fits the available data better than the ‘graveyard’ model previously envisaged, in which the liver was thought to sequester T cells already committed to apoptosis. Thus, the liver controls the size of the memory CD8+ T cell pool generated during a systemic immune response, by modulating the contraction phase of the effector CD8+ T cells. The liver carries out this function through a TLR-4 mediated mechanism.

The ability to enhance the formation of CD8+ memory T cells, without a large effect on the magnitude of the primary response, suggest that this immunoregulatory mechanism may be a therapeutic target.

### Example 11

### Affect of TLR-4 Induced T Cell Trapping on Memory Against H5N1 Influenza Vaccine

The central proposition, based on the data of the preceding examples, is that activated CD8+ T cells are trapped in the liver in a TLR-4 dependent manner and this trapping limits the size of the pool of circulating cells that form T cell memory. Interfering with this process should increase memory, and thus act as an adjunct to vaccination. In this experiment, this will be tested using a vaccine against an important human pathogen, avian influenza.

Groups of normal versus TLR-4 deficient mice (both described above) will be given the experimental H5N1 flu vaccine. The dose of vaccine will be titrated across five ten-fold steps, down to a dose that would normally generate no immunity. Flu-specific CD8+ T cells will be enumerated in the peripheral blood at the peak of the T cell response, using peptide-MHC tetramers. After six weeks, mice will be challenged with an attenuated recombinant strain of H5N1 influenza. The magnitude of the memory CD8+ T cell response will be measured using tetramers, and lung viral titer will be measured by real-time RT-PCR. Immediately prior to tissue harvest, mice will be bled to determine the level of aminotransaminase enzymes (AST and ALT) used to measure liver injury. The livers of these mice will be analyzed by H&E histology.

It is predicted that TLR-4 deficient mice will sequester fewer flu-specific CD8+ T cells in the liver; therefore, more will circulate in blood at the time of acute infection. This increased pool will give rise to more memory cells. When the primed mice are challenged, larger CD8+ T cell responses and more rapid suppression of viral RNA are expected. Conversely, less flu-associated liver injury is expected (Polakos et al., “Kupffer Cell-dependent Hepatitis Occurs During Influenza Infection,” Am J Pathol. 168(4):1169-78 (2006), which is hereby incorporated by reference in its entirety) in the TLR-4 deficient mice

This experiment will then be repeated using groups of wildtype mice who are administered the most-effective H5N1 vaccine titration, in combination with varying dosage schedules of the TLR-4 inhibitor eritoran (E5564) from Eisai Inc. Dosage schedules will be titrated to determine the lowest effective dose when administered at varying time points between days 0-15, 0-30, and 0-60 post-vaccination.

### Example 12

### Enhancement of Adoptive Immunotherapy Against Primary Tumors

Adoptive immunotherapy for cancer, and for virus infections in the context of bone marrow transplantation, is only moderately effective. Cell-tagging studies show that many of the activated T cells go to the liver. Therefore, the action of TLR-4 during adoptive immunotherapy will be transiently suppressed, and effects on T cell homing and anti-cancer effect will be measured.

Tumor-specific T cells will be isolated from resected malignant melanomas, or other immunogenic tumors and activated in vitro using antibodies against the T cell receptors plus cytokines. These T cells will be labeled with a radioactive tracer, and then injected into patients with multiple extrahepatic metastases. In Phase 1, all of the patients, and in Phase 2 half of the patients will additionally receive the therapeutic TLR-4 inhibitor eritoran (E5564) from Eisai, Inc., and the remainder will be given PBS as a placebo. These patients will be monitored for T cell localization and anti-tumor action.

The prediction is that if TLR-4 is temporarily inactivated, a smaller proportion of the activated T cells will localize to the liver. This will be assessed by detecting localization of the radioactive tracer. In phase 2, a therapeutic benefit may be observed, in terms of increased tumor shrinkage. Tumor size will be assessed by computerized axial tomography.

Although the invention has been described in detail for purposes of illustration, it is understood that such detail is solely for that purpose, and variations can be made therein by those skilled in the art without departing from the spirit and scope of the invention as defined in the claims that follow.

1. A method of inhibiting intrahepatic CD8+ T cell deletion comprising:
providing a Toll-like receptor-4 (TLR-4) inhibitor; and
administering the TLR-4 inhibitor to a subject in an amount effective to inhibit intrahepatic CD8+ T cell deletion.

2. The method of claim 1, wherein the TLR-4 inhibitor is selected from the group of an anti-TLR-4 antibody, a nucleic acid expressing antisense TLR-4 RNA, an aptamer that binds to TLR-4 and perturbs TLR-4 function, a nucleic acid encoding a ribozyme that cleaves TLR-4 mRNA, an antisense TLR-4 oligodeoxynucleotide, a protein sequence that corresponds to at least a portion of a receptor that binds to a TLR-4 ligand during TLR-4 signal transduction event, a non-TLR-4 polypeptide that inhibits TLR-4 function, and an inhibitory ligand that is a variant of a natural ligand of TLR-4.

3-8. (canceled)

9. The method according to claim 1, wherein said administering is carried out orally, topically, transdermally, parenterally, subcutaneously, intravenously, intramuscularly, intraperitoneally, by intracavitary or intravesical instillation, intranasally, intraocularly, intraarterially, intralesionally, or by application to mucous membranes.

10. The method according to claim 1, wherein the subject is a mammal.

11. The method according to claim 10, wherein the mammal is selected from the group of human, non-human primates, mouse, rat, guinea pig, rabbit, cat, dog, horse, cow, sheep, goat, pig.

12. A composition comprising:
a Toll-like receptor-4 (TLR-4) inhibitor; and
an immunogenic agent.

13. The composition according to claim 12, wherein the TLR-4 inhibitor is selected from the group of an anti-TLR-4 antibody, a nucleic acid expressing antisense TLR-4 RNA, an aptamer that binds to TLR-4 and perturbs TLR-4 function, a nucleic acid encoding a ribozyme that cleaves TLR-4 mRNA, an antisense TLR-4 oligodeoxynucleotide, a protein sequence that corresponds to at least a portion of a receptor that binds to a TLR-4 ligand during TLR-4 signal transduction event, a non-TLR-4 polypeptide that inhibits TLR-4 function, and an inhibitory ligand that is a variant of a natural ligand of TLR-4.

14-19. (canceled)

20. The composition according to claim 12, wherein the immunogenic agent is a polypeptide comprising a surface epitope of a T cell activating pathogen.

21. The composition according to claim 20, wherein the T cell activating pathogen is a bacterium, a virion, or parasite or an immunogenic cancer.

22. The composition according to claim 20, wherein the T cell activating pathogen is selected from the group of Listeria monocytogenes, Leishmania leishmaniasis, Chlamydia trachomatis, Mycobacterium tuberculosis, Influenza sp., Trypanosoma cruzi, Lentivirus sp., Hepacivirus sp., or an immunogenic cancer.

23. The composition according to claim 12 further comprising a pharmaceutically acceptable carrier.

24. The composition according to claim 23, wherein the composition is in the form of a vaccine.

25. The composition according to claim 12 further comprising an adjuvant.

26. A delivery vehicle comprising the composition according to claim 23.

27. A composition comprising:
activated CD8+ T cells; and
a Toll-like receptor-4 (TLR-4) inhibitor.

28. The composition according to claim 27 wherein the Toll-like receptor 4 inhibitor is selected from the group consisting of an anti-TLR-4 antibody, a nucleic acid expressing antisense TLR-4 RNA, an aptamer that binds to TLR-4 and perturbs TLR-4 function, a nucleic acid encoding a ribozyme that cleaves TLR-4 mRNA, an antisense TLR-4 oligodeoxynucleotide, a protein sequence that corresponds to at least a portion of a receptor that binds to a TLR-4 ligand during TLR-4 signal transduction event, a non-TLR-4 polypeptide that inhibits TLR-4 function, and an inhibitory ligand that is a variant of a natural ligand of TLR-4.

29-33. (canceled)

34. The composition according to claim 27, wherein the activated CD8+ T cells are isolated from a subject exposed to an immunogenic challenge.

35. The composition according to claim 34, where the immunogen used to induce the CD8+ T cells is selected from the group of Listeria monocytogenes, Leishmania leishmaniasis, Chlamydia trachomatis, Mycobacterium tuberculosis, Influenza sp., Trypanosoma cruzi, Lentivirus sp., Hepacivirus sp., or an immunogenic cancer.

36. The composition according to claim 34, wherein the subject is a mammal.

37. The composition according to claim 36, wherein the mammal is selected from the group of human, non-human primates, mouse, rat, guinea pig, rabbit, cat, dog, horse, cow, sheep, goat, pig.

38. The composition according to claim 27 further comprising a pharmaceutically acceptable carrier.

39. A delivery vehicle comprising the composition according to claim 38.

40. A method of enhancing a secondary immune response comprising:
providing a composition according to claim 12 or a combination of a TLR-4 inhibitor and an immunogenic agent; and
administering the composition or the combination to a subject in an amount effective to activate a T cell response while inhibiting intrahepatic deletion of activated T cells, thereby increasing the survival of memory cells to afford an enhanced secondary immune response to the immunogenic agent, T cell activating pathogen, or its equivalent.

41. The method according to claim 40 further comprising:
repeating said administering.

42. The method according to claim 40 further comprising:
administering a TLR-4 inhibitor following a delay after said administering the composition or the combination.

43-45. (canceled)

46. The method according to claim 40, wherein the T cell activating pathogen is a bacterium, a virion, or parasite, or an immunogenic cancer.

47. The method according to claim 40, wherein T cell activating pathogen is selected from the group of Listeria monocytogenes, Leishmania leishmaniasis, Chlamydia trachomatis, Mycobacterium tuberculosis, Influenza sp., Trypanosoma cruzi, Lentivirus sp., Hepacivirus sp., or an immunogenic cancer.

48. A method of enhancing a secondary immune response in an immuno-compromised subject comprising:
providing a composition according to claim 27 or a combination of TLR-4 inhibitor and activated CD8+ T cells; and
administering the composition or the combination to an immuno-compromised subject in an amount effective to promote survival of memory cells to afford enhanced secondary immune response to an immunogenic agent, T cell activating pathogen, or its equivalent.

49. The method according to claim 48 further comprising:
repeating said administering.

50. The method according to claim 48 further comprising:
administering a TLR-4 inhibitor following a delay after said administering the composition or the combination.

51-53. (canceled)

54. A method of enhancing a secondary immune response in a subject comprising:
administering to a subject an amount of a Toll-like receptor-4 (TLR-4) inhibitor that is effective to promote the survival of memory cells to afford enhanced secondary immune response to an immunogenic agent, T cell activating pathogen, or its equivalent.

55. The method according to claim 54 further comprising:
administering a vaccine comprising an immunogenic agent to the subject.

56. The method according to claim 55, wherein said administering the vaccine is carried out prior to said administering the TLR-4 inhibitor.

57. The method according to claim 55, wherein said administering the vaccine is carried out contemporaneously with said administering the TLR-4 inhibitor.

58. The method according to claim 55, wherein said administering the vaccine is carried out subsequent to said administering the TLR-4 inhibitor.

59. The method according to claim 55 further comprising repeating said administering the TLR-4 inhibitor.


SEQUENCE LISTING (1 sequence)

SEQ ID NO: 1 - protein, 8 aa, Artificial
  Antigenic peptide
SIINFEKL